(19) United States
(12) Patent Application Publication
(10) Pub. No.: US 2008/0234516 A1
(43) Pub. Date: Sep. 25, 2008

(54) Processes for preparing solid states of O-desmethylvenlafaxine succinate

(76) Inventors: Valerie Niddam-Hildesheim, Kadima (IL); Sharona Shachan-Tov, Kfar-Saba (IL); Yaron Shmuely, Hedera (IL); Tamar Nidam, Yehud (IL); Alexandr Jegorov, Dobra Voda (CZ)

Correspondence Address: KENYON & KENYON LLP, One Broadway, New York, NY, 10004, US

(21) Appl. No.: 12/075,833

(22) Filed: Mar. 14, 2008

Related U.S. Application Data
(60) Provisional application No. 60/918,176, filed on Mar. 14, 2007.

(51) Int. Cl. C07C 211/01

(52) U.S. Cl. 564/316

(57) ABSTRACT

Provided are processes for the preparation of amorphous O-desmethylvenlafaxine and for the preparation of crystalline forms I, II, III, and IV of O-desmethylvenlafaxine.

### CROSS REFERENCE TO RELATED APPLICATIONS

The present invention claims the benefit of the following U.S. Provisional Patent Application No. 60/918,176, filed Mar. 14, 2007. The contents of this application is incorporated herein by reference.

### FIELD OF THE INVENTION

The present invention is directed to processes for the preparation of solid states of O-desmethylvenlafaxine succinate.

### BACKGROUND OF THE INVENTION

Venlafaxine, (±)-1-[2-(Dimethylamino)-1-(4-ethyoxyphenyl) ethyl] cyclo-hexanol, having the following formula I, is the first of a class of anti-depressants. Venlafaxine acts by inhibiting re-uptake of norepinephrine and serotonin, and is an alternative to the tricyclic anti-depressants and selective re-uptake inhibitors.

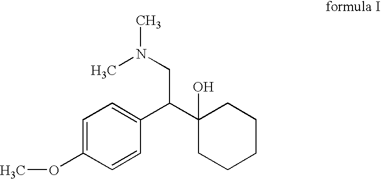

O-desmethylvenlafaxine, 4-[2-(dimethylamino)-1-(1-hydroxycyclohexyl)ethyl]phenol, having the following formula II

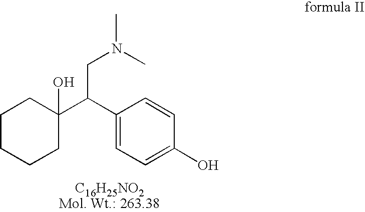

is a major metabolite of venlafaxine and has been shown to inhibit norepinephrine and serotonin uptake. Klamerus, K. J. et al., “Introduction of the Composite Parameter to the Pharmacokinetics of Venlafaxine and its Active O-Desmethyl Metabolite”, J. Clin. Phavmacol. 32:716-724 (1992).

O-desmethylvenlafaxine and processes for preparation thereof are described in U.S. Pat. Nos. 6,197,828 and 6,689,912, and in US 2005/0197392, which are incorporated herein by reference.

The succinate salt of O-desmethylvenlafaxine, chemically named 1-[2-(dimethylamino)-1-(4-phenol)ethyl]-cyclohexanol succinate, and having the following formula III

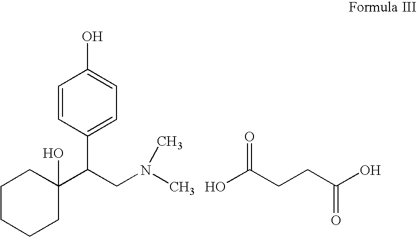

is described in U.S. Pat. No. 6,673,838. Also described in U.S. Pat. No. 6,673,838 are the amorphous form of O-desmethylvenlafaxine succinate and polymorphic forms of O-desmethylvenlafaxine succinate, therein referred to as Forms I, II, III and IV.

There is a need for additional processes for preparing the amorphous form and the polymorphic forms of O-desmethylvenlafaxine succinate, particularly those suitable for use on industrial scale.

### SUMMARY OF THE INVENTION

The present invention provides processes for the preparation of O-desmethylvenlafaxine succinate crystalline forms I, II, III and IV and O-desmethylvenlafaxine succinate amorphous form.

In one embodiment of the present invention there is provided a process of preparing crystalline O-desmethylvenlafaxine succinate form I comprising: combining O-desmethylvenlafaxine, succinic acid and a solvent to a slurry or suspension thereby forming crystalline O-desmethylvenlafaxine succinate form I, wherein the solvent is selected from the group consisting of: water; a mixture of water and either tween (1%), dimethylsulfoxide (DMSO), dimethyacetamide (DMA), toluene, dioxane, butanol, dichloromethane (DCM), hexane, ethylacetate, cyclohexanone, or methanol; methanol; a mixture of methanol and either hexane, cyclohexanone, acetonitrile, methylethyl ketone (MEK), toluene, acetone, dioxane, or xylene; a mixture of water, methanol and either isopropylalcohol, acetonitrile, MEK, toluene, 2-butanol or acetone; 1-propanol; t-butanol; 1-octanol; a mixture of isopropyl alcohol, acetic acid and heptane; iso amyl alcohol; tetrahydrofuran (THF); DMSO; a mixture of DMSO, MEK, and hexane; acetonitrile; a mixture of dimethylformamide (DMF) and MEK; and a mixture of DMA and MEK. Optionally, the mixture of water and toluene as a solvent may further contain a phase transfer catalyst, preferably aliquat 366. Preferably, in the above process O-desmethylvenlafaxine may be in the form of a free base or a succinate salt.

In another embodiment of the present invention there is provided a process of preparing crystalline O-desmethylvenlafaxine succinate form I comprising: heating to a temperature of about 60° C. to about 100° C. a mixture of O-desmethylvenlafaxine, succinic acid and a solvent to a slurry or suspension thereby forming crystalline O-desmethylvenlafaxine succinate form I, wherein the solvent is selected from the group consisting of: water; a mixture of water and either ethylacetate, hexane, DCM, or di-ethylene glycol; THF; and a mixture of ethylene glycol and hexane and wherein when the solvent is water heating is to about 90° C. Preferably, forming crystalline O-desmethylvenlafaxine succinate form I comprises cooling the slurry or suspension to about room temperature. Preferably, in the above process O-desmethylvenlafaxine may be in the form of a free base or a succinate salt.

In another embodiment of the present invention there is provided a process preparing crystalline O-desmethylvenlafaxine succinate form I comprising: a) providing a mixture of O-desmethylvenlafaxine and succinic acid; b) heating the mixture to melt; c) adding a solvent to the mixture; and d) precipitating O-desmethylvenlafaxine form I from the mixture. Preferably, the solvent is a C₅₋₈ alcohol, preferably an amyl alcohol, or a mixture of a C₄₋₇ ketone, preferably methylisobutyl ketone (MIBK), and water.

Another embodiment provides a process preparing crystalline O-desmethylvenlafaxine succinate form I comprising: providing a solution of O-desmethylvenlafaxine succinate in a solvent selected from the group consisting of; water; a mixture of water and either isopropyl alcohol, acetonitrile, toluene, ethylacetate, di-isopropylether, 1-propanol, t-butanol, 2-butanol, or MEK; and a mixture of methanol and ethylacetate; and precipitating crystalline O-desmethylvenlafaxine succinate form I from the solution, wherein when the solvent is a mixture of water and MEK the mixture is in a 2/4 ratio. The solution of O-desemethylvenlafaxine in a solvent may be obtained by sonicating a mixture of O-desmethylvenlafaxine base, a solvent and succinic acid. The solution of O-desmethylvenlfaxine succinate in a solvent may also be obtained by heating a mixture of O-desmethylvenlafaxine succinate and the solvent. Preferably, precipitating comprises cooling the solution, more preferably the solution is cooled to a temperature of about −5° C. to about 15° C., even more preferably to about 0° C. to about 10° C. If the above solution is a heated solution the cooling is preferably a two step process wherein the heated solution is first cooled to about room temperature and in a second step to a temperature of about −5° C. to about 15° C., more preferably of about 0° C. to about 10° C.

Another embodiment of the present invention provides a process preparing crystalline O-desmethylvenlafaxine succinate form I comprising: a) providing a solution of O-desmethylvenlafaxine succinate in a C₁₋₄ alcohol, preferably methanol; and b) precipitating crystalline O-desmethylvenlafaxine succinate form I by cooling to a temperature of about −5° C. to about 15° C. Preferably, cooling is carried out by thermal shock of adding the solution in a cooled solvent selected from the group consisting of a C₆₋₈ aromatic hydrocarbon, a C₆₋₈ hydrocarbon, a C₄₋₇ ester, a halogenated C₁₋₄ hydrocarbon, a C₃₋₈ ether, and acetone, wherein the cooled solvent is at a temperature of about −5° C. to about 15° C., preferably at about 0° C. to about 10° C.

In one embodiment of the present invention there is provided a process of preparing crystalline O-desmethylvenlafaxine succinate form II comprising: combining O-desmethylvenlafaxine, succinic acid and a solvent to a slurry or suspension thereby forming crystalline O-desmethylvenlafaxine succinate form II, wherein the solvent is selected from the group consisting of: a mixture of water and either a phase transfer catalyst, acetone, acetonitrile, polyethylene glycol, or methanol and a phase transfer catalyst; ethanol, preferably absolute ethanol; n-butanol; 2-ethoxyethanol; dichloroethane; buthylacetate; methylacetate; ethylacetate; dimethylcarbonate; ethyl lactate and saturated sodium chloride in water. Preferably, the phase transfer catalyst is sodium laurel sulfate (SLS). Preferably, when the solvent is a mixture of water and SLS (1%) forming O-desmethyl venlafaxine succinate form II comprises maintaining the slurry or suspension for about 3 hours. Preferably, when the solvent is 2-ethoxyethanol (cellosolve) forming O-desmethylvenlafaxine succinate form II comprises maintaining the slurry or suspension for a period of about 2 hours. Preferably, in the above process O-desmethylvenlafaxine may be in the form of a free base or a succinate salt.

In another embodiment of the present invention there is provided a process of preparing crystalline O-desmethylvenlafaxine succinate form II comprising: heating to a temperature of about 60° C. to about 70° C., preferably about 65° C., a mixture of O-desmethylvenlafaxine, succinic acid and water to a slurry or suspension thereby form crystalline O-desmethylvenlafaxine form II. Preferably, forming crystalline O-desmethylvenlafaxine succinate form II comprises cooling the the slurry or suspension to about room temperature. Preferably, in the above process O-desmethylvenlafaxine may be in the form of a free base or a succinate salt.

In yet another embodiment of the present invention there is provided a process of preparing crystalline O-desmethylvenlafaxine succinate form II comprising: exposing O-desmethyl venlafaxine succinate to solvent vapors for a period of time sufficient to obtain crystalline O-desmethylvenlafaxine succinate form II. Preferably, the solvent is selected from the group consisting of acetone, and a halogenated C₁₋₄ hydrocarbon, preferably DCM. Alternatively, O-desmethylvenlafaxine succinate is heated in a closed environment in the absence of a solvent.

Another embodiment provides a process for preparing crystalline O-desmethylvenlafaxine succinate form II comprising: a) providing a mixture of O-desmethylvenlafaxine base, succinic acid, and a solvent; and b) removing the solvent, preferably by azeotropic distillation, to obtain crystalline O-desmethylvenlafaxine succinate form II, wherein the solvent is selected from a mixture of water with a C₁₋₄ alcohol, preferably butanol, or a C₄₋₈ cyclic ether, preferably dioxane.

Another embodiment provides a process for preparing crystalline O-desmethylvenlafaxine succinate form II comprising: a) providing a solution of O-desmethylvenlafaxine succinate in a solvent selected from the group consisting of: n-butanol; 2-butanol; 1-propanol; ethanol; methyl isobutyl ketone (MIBK); dichlorobenzene; a mixture of water and methylethyl ketone (MEK); acetonitrile; and dioxane; and precipitating crystalline O-desmethylvenlaxine succinate form II from the solution, wherein when the solvent is acetonitrile precipating is carried out for about 1 hours to about 4 hours. The solution may be provided by heating a mixture of O-desmethylvenlafaxine succinate and a solvent or by adding succinic acid to a mixture of O-desmethylvenlafaxine base, water and a C₄₋₇ ketone. Precipitation of the crystalline form may be carried out by cooling the solution wherein cooling preferably comprises cooling of the solution in a first cooling step to about room temperature and a second cooling step to a temperature of about −5° C. to about 15° C., preferably at about 0° C. to about 10° C. to obtain crystalline O-desmethylvenlafaxine succinate form II. Precipitation may also be carried out by sonication of the solution to obtain crystalline O-desmethylvenlafaxine form II.

Another embodiment provides a process for preparing crystalline O-desmethylvenlafaxine form II comprising: a) providing a mixture of succinic acid and water; b) heating the mixture; and c) adding O-desmethylvenlafaxine base to the heated mixture; and d) cooling the heated mixture to obtain crystalline O-desmethylvenlafaxine succinate form II. Cooling preferably comprises cooling of the heated mixture in a first cooling step to about room temperature to about 35° C. and a second cooling step to a temperature of about −5° C. to about 15° C., preferably at about 0° C. to about 10° C.

In another embodiment of the present invention there is provided a process preparing O-desmethylvenlafaxine succinate form III comprising: combining O-desmethylvenlafaxine, succinic acid and a solvent to a slurry or suspension to thereby form crystalline O-desmethylvenlafaxine succinate form III, wherein the solvent is selected from the group consisting of: a mixture of ethanol and ether; di-isopropyl ether; butyl lactate; DCM; and a mixture of water, toluene and a phase transfer catalyst. Preferably, the phase transfer catalyst is aliquat 366. Preferably, in the above process O-desmethylvenlafaxine may be in the form of a free base or a succinate salt.

In yet another embodiment of the present invention there is provided a process preparing O-desmethylvenlafaxine succinate form III comprising: reacting O-desmethylvenlafaxine and succinic acid at a temperature of about 80-100° C. for more than 50 hours and cooling to room temperature to obtain crystalline O-desmethylvenlafaxine Form III.

Another embodiment provides a process preparing crystalline O-desmethylvenlafaxine succinate form III comprising: exposing crystalline forms I and II of O-desmethylvenlafaxine succinate to pressure to obtain crystalline O-desmethylvenlafaxine form III. Optionally, about 3-5 drops of solvent per about 300 grams of crystalline forms I and II of O-desmethylvenlafaxine are added prior to exposure to pressure. Preferably, the solvent is selected from the group consisting of water, a C₁₋₄ alcohol, and a C₃₋₇ ketone.

In another embodiment of the present invention there is provided a process preparing crystalline O-desmethylvenlafaxine succinate form III comprising: a) providing a mixture of O-desmethylvenlafaxine base, succinic acid and a solvent; and b) removing the solvent, preferably by azeotropic distillation, to obtain an oily substance; d) adding Methyl Ethyl Ketone (MEK) to the oily substance to form a mixture; and e) maintaining the mixture for a period of time sufficient to obtain crystalline O-desmethylvenlafaxine form III. Preferably the solvent is a mixture of a C₄₋₇ ketone and in water.

In another embodiment of the present invention there is provided a process preparing crystalline O-desmethylvenlafaxine succinate form III comprising: milling O-desmethylvenlafaxine to which about 1 drop of water or methanol per 100 mg O-desmethylvenlafaxine is added at room temperature. Preferably, milling is for a period of about 30 minutes to about 2 hours, more preferably for about 1 hour.

In yet another embodiment of the present invention there is provided a process preparing crystalline O-desmethylvenlafaxine succinate form IV comprising: exposing O-desmethylvenlafaxine succinate to vapors of a C₁₋₄ alcohol, preferably methanol, to obtain crystalline O-desmethylvenlafaxine form IV.

In another embodiment of the present invention there is provided a process preparing crystalline O-desmethylvenlafaxine succinate forms IV comprising: drying crystalline O-desmethylvenlafaxine succinate form I or II for a period of time sufficient to obtain crystalline O-desmethylvenlafaxine form IV. Preferably, drying comprises drying in the presence of a desiccant at a temperature of about 70° C. to about 80° C.

In yet another embodiment of the present invention there is provided a process preparing a mixture of crystalline O-desmethylvenlafaxine succinate forms I and II comprising: combining O-desmethylvenlafaxine, succinic acid and a solvent to a slurry or suspension thereby forming a mixture of crystalline O-desmethylvenlafaxine succinate forms I and II, wherein the solvent is selected from the group consisting of: a mixture of water and 1% sodium laurel sulfate (SLS); methyl tertbutyl ether (MTBE); n-amyl alcohol; 2-ethoxyethanol (cellosolve); isobutylacetate; a mixture of diethyleneglycol and methyl ethyl ketone (MEK); diethyl ether; a mixture of ethylene glycol and hexane; methyl ethyl ketone (MEK); 2-butanol; a mixture of n-butanol and methanol; a mixture of water and either acetone, dioxane or dioxane and methanol; and a mixture of methanol and either ethylacetate or polyethyleneglycol; and wherein when the solvent is 2-ethoxyethanol forming the crystalline O-desmethylvenlafaxine succinate form I and II comprises maintaining the suspension or slurry for a period of about 22 hours and when the solvent is a mixture of water and 1% SLS for a period of about 16 hours. Preferably, the slurry or suspension is heated to about 60° C. to about 70° C., preferably to about 65° C., when the solvent is a mixture of ethylene glycol and hexane. Preferably, in the above process O-desmethylvenlafaxine may be in the form of a free base or a succinate salt.

In another embodiment of the present invention there is provided a process preparing a mixture of crystalline O-desmethylvenlafaxine succinate forms I and II comprising: a) providing a mixture of O-desmethylvenlafaxine base, succinic acid, and a solvent; and b) removing the solvent, preferably by azeotropic distillation, to obtain crystalline O-desmethylvenlafaxine succinate forms I and II, wherein the solvent is a mixture of water with isopropyl alcohol or chloroform.

In another embodiment of the present invention there is provided a process preparing a mixture of crystalline O-desmethylvenlafaxine succinate forms I and II comprising: a) providing a mixture of O-desmethylvenlafaxine base in acetone b) heating the mixture; c) adding a solution of succinic acid in acetone to the heated mixture; d) maintaining the heated mixture for about 1 hour to 4 hours; and e) cooling the heated mixture to obtain a mixture of crystalline O-desmethylvenlafaxine forms I and II.

In yet another embodiment of the present invention there is provided a process preparing a mixture of crystalline O-desmethylvenlafaxine succinate forms I and II comprising: a) providing a suspension of O-desmethylvenlafaxine base and a solvent mixture of acetonitrile and water; b) adding succinic acid to the suspension to obtain a mixture; c) heating the mixture to obtain a solution; d) adding water to the solution; e) cooling slowly to about 30° C.; and D cooling in a second cooling step to a temperature of about 0-5° C. to obtain a mixture of crystalline O-desmethylvenlafaxine forms I and II.

In another embodiment of the present invention there is provided a process preparing a mixture of crystalline O-desmethylvenlafaxine succinate forms I and II comprising: a) providing O-desmethylvenlafaxine base and succinic acid in a solvent mixture to obtain a suspension; b) sonicating the suspension; and c) maintaining the suspension for a period of time sufficient to obtain a mixture of crystalline O-desmethylvenlafaxine forms I and II, wherein the solvent mixture is a mixture of water with a C₆₋₈ hydrocarbon.

In yet another embodiment of the present invention there is provided a process preparing a mixture of crystalline O-desmethylvenlafaxine succinate forms I and II comprising: exposing Form II to water vapors.

In another embodiment of the present invention there is provided a process preparing a mixture of crystalline O-desmethylvenlafaxine succinate forms II and III comprising: a) providing O-desmethylvenlafaxine base in a solvent to obtain a mixture; b) heating the mixture; c) adding succinic acid to the heated mixture; and d) cooling the mixture to room temperature to obtain a mixture of crystalline O-desmethylvenlafaxine succinate forms II and III, wherein the solvent is selected from methylisobuthyl keton (MIBK) and n-heptane.

In yet another embodiment of the present invention there is provided a process preparing a mixture of crystalline O-desmethylvenlafaxine succinate forms II and IV comprising: combining O-desmethylvenlafaxine, succinic acid and a solvent to a slurry or suspension thereby forming a mixture of crystalline O-desmethylvenlafaxine forms II and IV, wherein the solvent is selected from the group consisting of: a mixture of methanol with ethylacetate or isopropyl alcohol, and a halogenated C₁₋₄ hydrocarbon. Preferably, in the above process O-desmethylvenlafaxine may be in the form of a free base or a succinate salt.

In yet another embodiment of the present invention there is provided a process preparing amorphous O-desmethylvenlafaxine succinate comprising: reacting O-desmethylvenlafaxine and succinic acid at a temperature of about 80-100° C. for about 16 hours and cooling to room temperature to obtain amorphous O-desmethylvenlafaxine.

In another embodiment of the present invention there is provided a process preparing amorphous O-desmethylvenlafaxine succinate comprising: a) dissolving O-desmethylvenlafaxine in a C₁₋₄ alcohol, preferably t-butanol, and b) removing the solvent by lyophilizing or spray drying the solution to obtain amorphous O-desmethylvenlafaxine.

In another embodiment of the present invention there is provided a process preparing amorphous O-desmethylvenlafaxine succinate comprising: a) providing a mixture of O-desmethylvenlafaxine base and a C₁₋₄ alcohol; b) heating the mixture; c) adding a solution of succinic acid in a C₁₋₄ alcohol to the heated mixture; d) maintaining the heated mixture for about 1 hour to 4 hours; and e) cooling the heated mixture to obtain amorphous O-desmethylvenlafaxine.

In yet another embodiment of the present invention there is provided a process preparing amorphous O-desmethylvenlafaxine succinate comprising: a) providing O-desmethylvenlafaxine succinate and adding about 2 to 6 drops, preferably about 3 drops, of water per 300 gram of O-desmethylvenlafaxine succinate; and b) drying the solid at a temperature of about 120° C. to about 175° C., preferably about 150° C. for a period of time sufficient to obtain amorphous O-desmethylvenlafaxine.

### DETAILED DESCRIPTION OF THE INVENTION

The present invention provides processes for the preparation of O-desmethylvenlafaxine succinate crystalline forms I, II, III and IV and O-desmethylvenlafaxine succinate amorphous form.

As used herein, the term “room temperature” or “ambient temperature” refers to a temperature of about 18° C. to about 25° C.

As used herein crystalline O-desmethylvenlafaxine succinate from I is characterized by an X-ray powder diffraction pattern having characteristic peaks at 10.20, 14.91, 20.56, 22.13, 23.71, 24.60, and 25.79 degrees 2 theta (±0.2 degrees 2 theta), crystalline O-desmethylvenlafaxine succinate from II is characterized by an X-ray powder diffraction pattern having characteristic peaks at 13.18, 14.04, 14.35, 14.66, 16.68, 17.67, 19.24, 25.13, and 31.78 degrees 2 theta (+0.2 degrees 2 theta), crystalline O-desmethylvenlafaxine succinate from III is characterized by an X-ray powder diffraction pattern having characteristic peaks at 13.74, 22.55, and 32.42 degrees 2 theta (±0.2 degrees 2 theta), crystalline O-desmethylvenlafaxine succinate from IV is characterized an X-ray powder diffraction pattern having characteristic peaks at 11.29, 17.22, 19.64, 20.91, 21.61, 28.86, 29.80, 30.60, 36.85, and 37.70 degrees 2 theta (±0.2 degrees 2 theta).

In one embodiment of the present invention there is provided a process of preparing crystalline O-desmethylvenlafaxine succinate form I comprising: combining O-desmethylvenlafaxine, succinic acid and a solvent to a slurry or suspension thereby forming crystalline O-desmethylvenlafaxine succinate form I, wherein the solvent is selected from the group consisting of: water; a mixture of water and either tween (1%), dimethylsulfoxide (DMSO), dimethyacetamide (DMA), toluene, dioxane, butanol, dichloromethane (DCM), hexane, ethylacetate, cyclohexanone, or methanol; methanol; a mixture of methanol and either hexane, cyclohexanone, acetonitrile, methylethyl ketone (MEK), toluene, acetone, dioxane, or xylene; a mixture of water, methanol and either isopropylalcohol, acetonitrile, MEK, toluene, 2-butanol or acetone; 1-propanol; t-butanol; 1-octanol; a mixture of isopropyl alcohol, acetic acid and heptane; iso amyl alcohol; tetrahydrofuran (THF); DMSO; a mixture of DMSO, MEK, and hexane; acetonitrile; a mixture of dimethylformamide (DMF) and MEK; and a mixture of DMA and MEK. Optionally, the mixture of water and toluene as a solvent may further contain a phase transfer catalyst, preferably the phase transfer catalyst is selected from a tetrabutylammonium hydrogensulphate, tetrabutylammonium bromide, tetrabutylammonium chloride, tetrabutylammonium iodide, benzyltriethyl ammonium chloride, aliquot, quaternary ammonium salt, quaternary phosphonium salt or crown ether, more preferably aliquat 366. Preferably, in the above process O-desmethylvenlafaxine may be in the form of a free base or a succinate salt. Preferably, the ratio of the water and any of the above organic solvents is from 2:10 to 7:1.5, more preferably from 2:10 to 2:8. Preferably the mixture of methanol and any of the above organic solvents is from 2:10 to 4:10, more preferably from 2:8 to 2:6. Preferably, forming crystalline O-desmethylvenlafaxine succinate form I comprises maintaining the slurry or suspension at about room temperature for a period of about 4 hours to about 11 days, more preferably from about 6 hours to about 7 days, even more preferably from about 16 hours to about 72 hours. Without wishing to be bound by theory the some of the O-desmethylvenlafaxine in the slurry or suspension may dissolve in the succininc acid and subsequently precipitates as therefrom as crystalline O-desmethylvenlafaxine succinate form I. When the mixture of solvents is a mixture of isopropanol, acetic acid and heptane or a mixture of MEK with either DMF or DMA both heptane and MEK are an anti-solvent.

In another embodiment of the present invention there is provided a process of preparing crystalline O-desmethylvenlafaxine succinate form I comprising: heating to a temperature of about 60° C. to about 100° C., preferably to about 60° C. to about 90° C., a mixture of O-desmethylvenlafaxine, succinic acid and a solvent to a slurry or suspension thereby forming crystalline O-desmethylvenlafaxine succinate form I, wherein the solvent is selected from the group consisting of: water; a mixture of water and either ethylacetate, hexane, DCM, or di-ethylene glycol; THF; and a mixture of ethylene glycol and hexane and wherein when the solvent is water heating is to about 90° C. Preferably, forming crystalline O-desmethylvenlafaxine succinate form I comprises cooling the slurry or suspension to about room temperature. Preferably, in the above process O-desmethylvenlafaxine may be in the form of a free base or a succinate salt. Preferably, forming crystalline O-desmethylvenlafaxine succinate form I comprises heating the slurry or suspension for a period of about 5.5 hours to about 6 days, more preferably from about 24 hours to about 72 hours. In this process the heated slurry or suspension is preferably cooled to about room temperature to obtain crystalline O-desemthylvenlafaxine succinate form I. Without wishing to be bound by theory the some of the O-desmethylvenlafaxine in the slurry or suspension may dissolve in the succininc acid and subsequently precipitates as therefrom as crystalline O-desmethylvenlafaxine succinate form I. When the solvent is a mixture of ethylene glycol and hexane, hexane is used as an anti-solvent.

In another embodiment of the present invention there is provided a process preparing crystalline O-desmethylvenlafaxine succinate form I comprising: a) providing a mixture of O-desmethylvenlafaxine and succinic acid; b) heating the mixture to melt; c) adding a solvent to the mixture; and d) precipitating O-desmethylvenlafaxine form I from the mixture. Preferably, the solvent is a C₅₋₈ alcohol, preferably an amyl alcohol, or a mixture of a C₄₋₇ ketone, preferably methylisobutyl ketone (MIBK), and water. Preferably heating is to a temperature of about 100° C. to about 150° C., more preferably to about 110° C. to about 130° C. Preferably, precipitating comprises cooling the mixture to about room temperature.

Another embodiment provides a process preparing crystalline O-desmethylvenlafaxine succinate form I comprising: providing a solution of O-desmethylvenlafaxine succinate in a solvent selected from the group consisting of; water; a mixture of water and either isopropyl alcohol, acetonitrile, toluene, ethylacetate, di-isopropylether, 1-propanol, t-butanol, 2-butanol, or MEK; and a mixture of methanol and ethylacetate; and precipitating crystalline O-desmethylvenlafaxine succinate form I from the solution, wherein when the solvent is a mixture of water and MEK the mixture is in a 2/4 ratio. The solution of O-desemethylvenlafaxine in a solvent may be obtained by sonicating a mixture of O-desmethylvenlafaxine base, a solvent and succinic acid, preferably at about 70% amplitude for a period of about 2.5 minutes to about 15 minutes, more preferably for about 10 minutes to about 15 minutes. This sonication of the mixture of O-desmethylvenlafaxine base, a solvent and succinic acid may result in an increase in temperature to about 45° C. to about 55° C., preferably to about 50° C. The solution of O-desmethylvenlfaxine succinate in a solvent may also be obtained by heating a mixture of O-desmethylvenlafaxine succinate and the solvent, preferably to about reflux. Preferably, precipitating comprises cooling the solution, more preferably the solution is cooled to a temperature of about −5° C. to about 15° C., even more preferably to about 0° C. to about 10° C. If the above solution is a heated solution the cooling is preferably a two step process wherein the heated solution is first cooled to about room temperature and in a second step to a temperature of about −5° C. to about 15° C., more preferably of about 0° C. to about 10° C. The solution obtained by sonication is preferably cooled to about room temperature and subsequently maintained at that temperature for a period of about 15 minutes to about 16 hours, more preferably for about 8 hours to about 16 hours.

Another embodiment of the present invention provides a process preparing crystalline O-desmethylvenlafaxine succinate form I comprising: a) providing a solution of O-desmethylvenlafaxine succinate in a C₁₋₄ alcohol, preferably methanol; and b) precipitating crystalline O-desmethylvenlafaxine succinate form I by cooling to a temperature of about −5° C. to about 15° C. Preferably, cooling is carried out by thermal shock of adding the solution in a cooled solvent selected from the group consisting of a C₆₋₈ aromatic hydrocabon, a C₆₋₈ hydrocarbon, a C₄₋₇ ester, a halogenated C₁₋₄ hydrocarbon, a C₃₋₈ ether, and acetone, wherein the cooled solvent is at a temperature of about −5° C. to about 15° C., preferably at about 0° C. to about 10° C. Preferably, the C₆₋₈ aromatic hydrocarbon is toluene, the C₆₋₈ hydrocarbon is hexane, the C₄₋₇ ester is ethylacetate, the halogenated C₁₋₄ hydrocarbon is dichloromethane, the C₃₋₈ ether is methyl tertbutyl ether (MTBE).

In one embodiment of the present invention there is provided a process of preparing crystalline O-desmethylvenlafaxine succinate form II comprising: combining O-desmethylvenlafaxine, succinic acid and a solvent to a slurry or suspension thereby forming crystalline O-desmethylvenlafaxine succinate form II, wherein the solvent is selected from the group consisting of: a mixture of water and either a phase transfer catalyst, acetone, acetonitrile, polyethylene glycol, or methanol and a phase transfer catalyst; ethanol, preferably absolute ethanol; n-butanol; 2-ethoxyethanol; dichloroethane; buthylacetate; methylacetate; ethylacetate; dimethylcarbonate; ethyl lactate and saturated sodium chloride in water. Preferably, the phase transfer catalyst is sodium laurel sulfate (SLS). Preferably, when the solvent is a mixture of water and SLS (1%) forming O-desmethyl venlafaxine succinate form II comprises maintaining the slurry or suspension for about 3 hours. Preferably, when the solvent is 2-ethoxyethanol (cellosolve) forming O-desmethylvenlafaxine succinate form II comprises maintaining the slurry or suspension for a period of about 2 hours. Preferably, in the above process O-desmethylvenlafaxine may be in the form of a free base or a succinate salt. Preferably, in the mixture of water with either acetone, acetonitrile, methanol, or polyethylene glycol the ratio is from 1:1 to 2:10, more preferably from 2:8 to 2:10, even more preferably from about 3:9 to 2:10. Preferably, forming crystalline O-desmethylvenlafaxine succinate form II comprises maintaining the slurry or suspension at about room temperature for a period of about 2 hours to about 4 days, more preferably from about 6 hours to about 22.5 hours, even more preferably from about 12 hours to about 20 hours.

In another embodiment of the present invention there is provided a process of preparing crystalline O-desmethylvenlafaxine succinate form II comprising: heating to a temperature of about 60° C. to about 70° C., preferably about 65° C., a mixture of O-desmethylvenlafaxine, succinic acid and water to a slurry or suspension thereby form crystalline O-desmethylvenlafaxine form II. Preferably, forming crystalline O-desmethylvenlafaxine succinate form II comprises cooling the the slurry or suspension to about room temperature. Preferably, in the above process O-desmethylvenlafaxine may be in the form of a free base or a succinate salt.

In yet another embodiment of the present invention there is provided a process of preparing crystalline O-desmethylvenlafaxine succinate form II comprising: exposing O-desmethylvenlafaxine succinate to solvent vapors for a period of time sufficient to obtain crystalline O-desmethylvenlafaxine succinate form II. Preferably, the solvent is selected from the group consisting of acetone, and a halogenated C₁₋₄ hydrocarbon, preferably DCM. Alternatively, O-desmethylvenlafaxine succinate is heated in a closed environment in the absence of a solvent. Preferably, exposing or heating is at a temperature of about 65° C. to about 75° C., more preferably to about 70° C. for a period of about 48 hours to about 4 days, more preferably for about 72 hours.

Another embodiment provides a process for preparing crystalline O-desmethylvenlafaxine succinate form II comprising: a) providing a mixture of O-desmethylvenlafaxine base, succinic acid, and a solvent; and b) removing the solvent, preferably by azeotropic distillation, to obtain crystalline O-desmethylvenlafaxine succinate form II, wherein the solvent is selected from a mixture of water with a C₁₋₄ alcohol, preferably butanol, or a C₄₋₈ cyclic ether, preferably dioxane. Preferably, azeotropic destillation is at about 80° C. Preferably, cooling is performed to obtain the crystalline form of O-desmethylvenlafaxine succinate to about room temperature, and maintained at this temperature for a period of about 16 hours to about 48 hours.

Another embodiment provides a process for preparing crystalline O-desmethylvenlafaxine succinate form II comprising: a) providing a solution of O-desmethylvenlafaxine succinate in a solvent selected from the group consisting of: n-butanol; 2-butanol; 1-propanol; ethanol; methyl isobutyl ketone (MIBK); dichlorobenzene; a mixture of water and methylethyl ketone (MEK); acetonitrile; and dioxane; and precipitating crystalline O-desmethylvenlaxine succinate form II from the solution, wherein when the solvent is acetonitrile precipating is carried out for about 1 hours to about 4 hours . The solution may be provided by heating, preferably to reflux, a mixture of O-desmethylvenlafaxine succinate and the said solvent or by adding succinic acid to a mixture of O-desmethylvenlafaxine base, water and a C₄₋₇ ketone, preferably MEK. Precipitation of the crystalline form may be carried out by cooling the solution wherein cooling preferably comprises cooling of the solution in a first cooling step to about room temperature and a second cooling step to a temperature of about −5° C. to about 15° C., preferably at about 0° C. to about 10° C., most preferably to about 5° C., to obtain crystalline O-desmethylvenlafaxine succinate form II. Precipitation may also be carried out by sonication of the solution to obtain crystalline O-desmethylvenlafaxine form II. Sonication may be carried out on the clear solution of O-desmethylvenlafaxine succinate in water and MEK for a period of about 15 minutes to about 20 minutes, preferably about 18 minutes, at about 40% amplitude.

Another embodiment provides a process for preparing crystalline O-desmethylvenlafaxine form II comprising: a) providing a mixture of succinic acid and water; b) heating the mixture; and c) adding O-desmethylvenlafaxine base to the heated mixture; and d) cooling the heated mixture to obtain crystalline O-desmethylvenlafaxine succinate form II. Cooling preferably comprises cooling of the heated mixture in a first cooling step to about room temperature to about 35° C. and a second cooling step to a temperature of about −5° C. to about 15° C., preferably at about 0° C. to about 10° C., more preferably to about 5° C. Heating the mixture of succinic acid and water is preferably to a temperature of about 50° C. to about 60° C., more preferably to about 55° C. The addition of O-desmethylvenlafaxine to such heated mixture may be carried out in portions.

In another embodiment of the present invention there is provided a process preparing O-desmethylvenlafaxine succinate form III comprising: combining O-desmethylvenlafaxine, succinic acid and a solvent to a slurry or suspension to thereby form crystalline O-desmethylvenlafaxine succinate form III, wherein the solvent is selected from the group consisting of: a mixture of ethanol and ether; di-isopropyl ether; butyl lactate; DCM; and a mixture of water, toluene and a phase transfer catalyst. Preferably, the phase transfer catalyst is aliquat 366. Preferably, in the above process O-desmethylvenlafaxine may be in the form of a free base or a succinate salt. The mixture of ethanol and ether is preferably in a ratio of about 28:8, and the mixture of water and toluene is preferably in a ratio of about 2:8. Preferably, forming crystalline O-desmethylvenlafaxine succinate form III comprises maintaining the slurry or suspension at about room temperature for a period of about 18 hours to about 6 days, more preferably of about 18 hours to about 24 hours.

In yet another embodiment of the present invention there is provided a process preparing O-desmethylvenlafaxine succinate form III comprising: reacting O-desmethylvenlafaxine and succinic acid at a temperature of about 80-100° C. for more than 50 hours and cooling to room temperature to obtain crystalline O-desmethylvenlafaxine Form III. Heating to about 80° C.-100° C. as above may preferably be for a period of about 50 hours to about 72 hours, more preferably about 54.5 hours.

Another embodiment provides a process preparing crystalline O-desmethylvenlafaxine succinate form III comprising: exposing crystalline forms I and II of O-desmethylvenlafaxine succinate to pressure to obtain crystalline O-desmethylvenlafaxine form III. Optionally, about 3-5 drops of solvent per about 300 grams of crystalline forms I and II of O-desmethylvenlafaxine are added prior to exposure to pressure. Preferably, the solvent is selected from the group consisting of water, a C₁₋₄ alcohol, and a C₃₋₇ ketone. Preferably, the C₁₋₄ alcohol is methanol, and the C₃₋₇ ketone is acetone. A pressure of about 8 to about 12, preferably about 10 tons may be applied to the solid material for a period of about 30 minutes to about 2 hours to obtain the crystalline O-desmethylvenlafaxine succinate form III.

In another embodiment of the present invention there is provided a process preparing crystalline O-desmethylvenlafaxine succinate form III comprising: a) providing a mixture of O-desmethylvenlafaxine base, succinic acid and a solvent; and b) removing the solvent, preferably by azeotropic distillation, to obtain an oily substance; d) adding Methyl Ethyl Ketone (MEK) to the oily substance to form a mixture; and e) maintaining the mixture for a period of time sufficient to obtain crystalline O-desmethylvenlafaxine form III. Preferably the solvent is a mixture of a C₄₋₇ ketone and in water. Preferably the C₄₋₇ ketone is methylethyl ketone (MEK). Preferably, azeotropic destination is at about 80° C. Preferably, cooling is performed to obtain the crystalline form of O-desmethylvenlafaxine succinate to about room temperature, and maintained at this temperature for a period of about 16 hours to about 48 hours. The mixture in step e) is preferably maintained at about room temperature for a period of about 4 days to about 12 days, more preferably about 9 days.

In another embodiment of the present invention there is provided a process preparing crystalline O-desmethylvenlafaxine succinate form III comprising: milling O-desmethylvenlafaxine to which about 1 drop of water or methanol per 100 mg O-desmethylvenlafaxine is added at room temperature. Preferably, milling is for a period of about 30 minutes to about 2 hours, more preferably for about 1 hour.

In yet another embodiment of the present invention there is provided a process preparing crystalline O-desmethylvenlafaxine succinate form IV comprising: exposing O-desmethylvenlafaxine succinate to vapors of a C₁₋₄ alcohol, preferably methanol, to obtain crystalline O-desmethylvenlafaxine form IV. Preferably, exposing O-desmethylvenlafaxine succinate to vapors is at about 65° C. to about 75° C., more preferably to about 70° C. for a period of about 48 hours to about 72 hours, more preferably for about 72 hours.

In another embodiment of the present invention there is provided a process preparing crystalline O-desmethylvenlafaxine succinate forms IV comprising: drying crystalline O-desmethylvenlafaxine succinate form I or II for a period of time sufficient to obtain crystalline O-desmethylvenlafaxine form IV. Preferably, drying comprises drying in the presence of a desiccant at a temperature of about 70° C. to about 80° C., more preferably at 80° C. and at about 0% relative humidity. The desiccant is preferably P₂O₅. Preferably, the crystalline O-desmethylvenlafaxine succinate form I or II are dried for a period of about 7 days to about 21 days, more preferably for about 7 days to about 17 days.

In yet another embodiment of the present invention there is provided a process preparing a mixture of crystalline O-desmethylvenlafaxine succinate forms I and II comprising: combining O-desmethylvenlafaxine, succinic acid and a solvent to a slurry or suspension thereby forming a mixture of crystalline O-desmethylvenlafaxine succinate forms I and II, wherein the solvent is selected from the group consisting of: a mixture of water and 1% sodium laurel sulfate (SLS); methyl tertbutyl ether (MTBE); n-amyl alcohol; 2-ethoxyethanol (cellosolve); isobutylacetate; a mixture of diethyleneglycol and methyl ethyl ketone (MEK); diethyl ether; a mixture of ethylene glycol and hexane; methyl ethyl ketone (MEK); 2-butanol; a mixture of n-butanol and methanol; a mixture of water and either acetone, dioxane or dioxane and methanol; and a mixture of methanol and either ethylacetate or polyethyleneglycol; and wherein when the solvent is 2-ethoxyethanol forming the crystalline O-desmethylvenlafaxine succinate forms I and II comprises maintaining the suspension or slurry for a period of about 22 hours and when the solvent is a mixture of water and 1% SLS for a period of about 16 hours. Preferably, the slurry or suspension is heated to about 60° C. to about 70° C., preferably to about 65° C., when the solvent is a mixture of ethylene glycol and hexane. Preferably, in the above process O-desmethylvenlafaxine may be in the form of a free base or a succinate salt. Preferably, the slurry or suspension is maintained for a period of about 16 hours to about 48 hours, more preferably for about 18 hours to about 24 hours at about room temperature. The heated slurry or suspension in ethylene glycol and hexane is preferably heated for a period of about 16 hours to about 24 hours and after cooling to about room temperature is maintained for a period of about 3 to 7 days, preferably about 5 days at about room temperature. When the solvent is a mixture of water and acetone the slurry or suspension is maintained at room temperature for a period of about 30 minutes to about 2 hours, preferably about 1 hour and subsequently at about 0° C. to about 10° C., preferably about 5° C., for a period of about 30 minutes to about 2 hours, preferably about 1 hour.

In another embodiment of the present invention there is provided a process preparing a mixture of crystalline O-desmethylvenlafaxine succinate forms I and II comprising: a) providing a mixture of O-desmethylvenlafaxine base, succinic acid, and a solvent; and b) removing the solvent, preferably by azeotropic distillation, to obtain crystalline O-desmethylvenlafaxine succinate forms I and II, wherein the solvent is a mixture of water with isopropyl alcohol or chloroform. Preferably, azeotropic destination is at about 80° C. to about 105° C. for a period of about 1.5 hours to about 5 hours. Preferably, after cooling to about room temperature the obtained material is maintained at this temperature for a period of about 2.5 hours to about 16 hours.

In another embodiment of the present invention there is provided a process preparing a mixture of crystalline O-desmethylvenlafaxine succinate forms I and II comprising: a) providing a mixture of O-desmethylvenlafaxine base in acetone b) heating the mixture; c) adding a solution of succinic acid in acetone to the heated mixture; d) maintaining the heated mixture for about 1 hour to 4 hours; and e) cooling the heated mixture to obtain a mixture of crystalline O-desmethylvenlafaxine forms I and II. Preferably, the mixture in step b) is heated to about 50° C. to about 60° C., more preferably to about 55° C. The addition of the heated solution of succininc acid in acetone may be carried out dropwise, after which addition, the mixture may be heated for another about 1 hour to about 2 hours, preferably about 2 hours at such temperature. Cooling may be carried out to a temperature of about −5° C. to about 10° C., preferably about 0° C. to about 5° C. for about 2 hours to about 4 hours.

In yet another embodiment of the present invention there is provided a process preparing a mixture of crystalline O-desmethylvenlafaxine succinate forms I and II comprising: a) providing a suspension of O-desmethylvenlafaxine base and a solvent mixture of acetonitrile and water; b) adding succinic acid to the suspension to obtain a mixture; c) heating the mixture to obtain a solution; d) adding water to the solution; e) cooling slowly to about 30° C.; and f) cooling in a second cooling step to a temperature of about 0-5° C. to obtain a mixture of crystalline O-desmethylvenlafaxine forms I and II. Preferably, the water and acetonitrile mixture is in a ratio of 2:9.

In another embodiment of the present invention there is provided a process preparing a mixture of crystalline O-desmethylvenlafaxine succinate forms I and II comprising: a) providing O-desmethylvenlafaxine base and succinic acid in a solvent mixture to obtain a suspension; b) sonicating the suspension; and c) maintaining the suspension for a period of time sufficient to obtain a mixture of crystalline O-desmethylvenlafaxine forms I and II, wherein the solvent mixture is a mixture of water with a C₆₋₈ hydrocarbon. Preferably, the C₆₋₈ hydrocarbon is heptane. The suspension is preferably sonicated for about 5 minutes to about 12 minutes, preferably 10 minutes, at about 70% amplitude. To so obtained suspension may be maintained at about room temperature for a period of about 12 hours to about 18 hours, preferably about 16 hours.

In yet another embodiment of the present invention there is provided a process preparing a mixture of crystalline O-desmethylvenlafaxine succinate forms I and II comprising: exposing Form II to water vapors. Preferably, exposing O-desmethylvenlafaxine succinate to vapors is at a temperature of about 65° C. to about 75° C., more preferably at about 70° C., for a period of about 60 hours to about 84 hours, more preferably about 72 hours.

In another embodiment of the present invention there is provided a process preparing a mixture of crystalline O-desmethylvenlafaxine succinate forms II and III comprising: a) providing O-desmethylvenlafaxine base in a solvent to obtain a mixture; b) heating the mixture; c) adding succinic acid to the heated mixture; and d) cooling the mixture to room temperature to obtain a mixture of crystalline O-desmethylvenlafaxine succinate forms II and III, wherein the solvent is selected from methylisobuthyl keton (MIBK) and n-heptane. Heating is preferably to a temperature of about 60° C. to about 70° C., more preferably to about 65° C. for a period of about 12 hours to about 18 hours.

In yet another embodiment of the present invention there is provided a process preparing a mixture of crystalline O-desmethylvenlafaxine succinate forms II and IV comprising: combining O-desmethylvenlafaxine, succinic acid and a solvent to a slurry or suspension thereby forming a mixture of crystalline O-desmethylvenlafaxine forms II and IV, wherein the solvent is selected from the group consisting of: a mixture of methanol with ethylacetate or isopropyl alcohol, and a halogenated C₁₋₄ hydrocarbon, preferably DCM. Preferably, in the above process O-desmethylvenlafaxine may be in the form of a free base or a succinate salt. Preferably, forming the mixture of crystalline O-desmethylvenlafaxine succinate forms II and IV comprises maintaining the slurry or suspension for a period of about 60 hours to about 84 hours, more preferably for about 72 hours at about room temperature. When the solvent is DCM or a mixture of isopropanol and methanol the slurry or suspension is preferably maintained at a temperature of about 60° C. to about 70° C., more preferably of about 65° C.

In yet another embodiment of the present invention there is provided a process preparing amorphous O-desmethylvenlafaxine succinate comprising: reacting O-desmethylvenlafaxine and succinic acid at a temperature of about 80-100° C. for about 16 hours and cooling to room temperature to obtain amorphous O-desmethylvenlafaxine.

In another embodiment of the present invention there is provided a process preparing amorphous O-desmethylvenlafaxine succinate comprising: a) dissolving O-desmethylvenlafaxine in a C₁₋₄ alcohol, preferably t-butanol, and b) removing the solvent by lyophilizing or spray drying the solution to obtain amorphous O-desmethylvenlafaxine.

Spray-drying broadly refers to processes involving breaking up liquid mixtures into small droplets, preferably, by atomization, and rapidly removing solvent from the mixture. In a typical spray-drying apparatus, there is a strong driving force for evaporation of solvent from the droplets, which may be provided by a heated drying gas. Spray-drying processes and equipment are described in Perry's CHEMICAL ENGINEER's HANDBOOK, pgs. 20-54 to 20-57 (Sixth Edition 1984).

By way of non-limiting example only, the typical spray-drying apparatus comprises a drying chamber, an atomizer for atomizing a solvent containing feed into the drying chamber, a source of heated drying gas that flows into the drying chamber to remove solvent from the atomized solvent containing feed, an outlet for the products of drying, and a product collector, located downstream of the drying chamber. Examples of such apparatuses include Niro Models PSD-1, PSD-2, and PSD-4 (Niro A/S, Soeborg, Denmark). Typically, the product collector includes a cyclone connected to the drying apparatus. In the cyclone, the particles produced during spray-drying are separated from the drying gas and evaporated solvent, allowing the particles to be collected. A filter may also be used to separate and collect the particles produced by spray-drying. The process of the invention is not limited to the use of such drying apparatuses as described above.

In another embodiment of the present invention there is provided a process preparing amorphous O-desmethylvenlafaxine succinate comprising: a) providing a mixture of O-desmethylvenlafaxine base and a C₁₋₄ alcohol; b) heating the mixture; c) adding a solution of succinic acid in a C₁₋₄ alcohol to the heated mixture; d) maintaining the heated mixture for about 1 hour to 4 hours; and e) cooling the heated mixture to obtain amorphous O-desmethylvenlafaxine. Heating is preferably to about 50° C. to about 60° C., more preferably to about 55° C. and cooling is preferably to about −5° C. to about 10° C., more preferably to about 0° C. to about 5° C. Preferably the C₁₋₄ alcohol is ethanol or methanol.

In yet another embodiment of the present invention there is provided a process preparing amorphous O-desmethylvenlafaxine succinate comprising: a) providing O-desmethylvenlafaxine succinate and adding about 2 to 6 drops, preferably about 3 drops, of water per 300 gram of O-desmethylvenlafaxine succinate; and b) drying the solid at a temperature of about 120° C. to about 175° C., preferably about 150° C. for a period of time sufficient to obtain amorphous O-desmethylvenlafaxine. Preferably, drying is for a period of about 12 hours to about 18 hours, more preferably about 16 hours.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

### EXAMPLES

### Preparation ODV Succinate Salt Form I

### Examples 1 to 43

### Preparing ODV Succinate Form I Using a Slurry Method.

### General Process for Slurry at Room Temperature

To a 100 ml flask equipped with a magnetic stirrer and condenser, were added ODV base and the solvent mixture. The mixture was stirred at room temperature for few minutes and succinic acid (1.1 equivalent) was added. The mixture was stirred at room temperature. Then the solid was filtered under reduced pressure, washed and dried in a vacuum oven overnight at 50° C.

| | | | Vol | | | | |
|---|---|---|---|---|---|---|---|
| | | | (ml/g of ODV) | | | | |
| | Solvent | Ratio | [Please confirm] | Time | | Sample | Remarks |
| 1 | H₂O | | 10 | 20 | h | Wet + dry | |
| 2 | H₂O | | 10 | 72 | h | Wet + dry | |
| 3 | H₂O | | 3 | 24 | h | Wet + dry | |
| 4 | H₂O + 1% tween | | 6 | 16 | h | Wet | |
| 5 | DMSO/H₂O | 1.5/7 | 8 | 16 | h | Wet + dry | Washed with MEK |
| 6 | DMA/H₂O | 3/7 | 8 | 16 | h | Wet + dry | Washed with MEK |
| 7 | Toluene/H₂O/aliquat | 8/2/0.1 eq | | 6 | days | Wet | |
| | 366 | | | | | | |
| 8 | Dioxane/H₂O | 10/2 | 12 | 24 | h | Dry | |
| 9 | BuOH/H₂O | 8/2 | 10 | 24 | h | Wet | |
| 10 | CH₂Cl₂/H₂O | 8/2 | 10 | 16 | h | Wet | |
| 11 | Hexane/H₂O | 8/2 | 10 | 16 | h | Wet | |
| 12 | EtOAc/H₂O | 8/2 | 10 | 16 | h | Wet | |
| 13 | Cyclohexanone/H₂O | 8/2 | 10 | 16 | h | Wet | |
| 14 | MeOH/H₂O/1% tween | 10/2 | 12 | 24-48 | h | Wet | |
| 15 | MeOH | | 5 | 20 | h | Wet + dry | Washed with MEK |
| 16 | Hexane/MeOH | 8/2 | 10 | 9 | days | Wet + dry | |
| 17 | Cyclohexanone/MeOH | 8/2 | 10 | 16 | h | Wet | |
| 18 | ACN/MeOH | 8/2 | 10 | 4 | days | Wet + dry | |
| 19 | MEK/MeOH | 8/2 | 10 | 72 | h | Wet + dry | |
| 20 | Toluene/MeOH | 8/2 | 10 | 72 | h | Wet + dry | |
| 21 | Toluene/MeOH | 5/2 | 7 | 6 | h | Wet + dry | |
| 22 | Acetone/MeOH | 8/2 | 10 | 48 | h | wet | |
| 23 | Dioxane/MeOH | 8/2 | 10 | 48 | h | wet | |
| 24 | Dioxane/MeOH | 5/1 | 12 | 72 | h | Wet + dry | |
| 25 | Xylene/MeOH | 5/2 | 7 | 7 | h | Wet + dry | |
| 26 | IPA/MeOH/H₂O | 6/2/2 | 10 | 48 | h | Wet | |
| 27 | ACN/MeOH/H₂O | 6/2/2 | 10 | 72 | h | Wet | |
| 28 | MEK/MeOH/H₂O | 6/2/2 | 10 | 72 | h | wet | |
| 29 | Toluene/MeOH/H₂O | 6/2/2 | 10 | 72 | h | Wet + dry | |
| 30 | 2-BuOH/MeOH/H₂O | 6/2/2 | 10 | 72 | h | Wet + dry | |
| 31 | Acetone/MeOH/H₂O | 6/2/2 | 10 | 3-4 | days | wet | |
| 32 | 1-PrOH | | 5 | 20 | h | Wet + dry | |
| 33 | t-BuOH | | 5 | 18 | h | Wet + dry | |
| 34 | 1-Octanol | | 5 | 18 | h | Wet + dry | |
| 35 | IPA/AcOH/Heptane | 10/0.4/10 | 20 | 4 | h | Wet + dry | Heptane as anti |
| | | | | | | | solvent |
| 36 | Iso Amyl alcohol | | 10 | 24 | h | Wet + dry | * Adding citric acid |
| | | | | | | | solution 10%, 5 ml |
| | | | | | | | * Cooled to 5° C., 9 h |
| 37 | Iso-Amyl Alcohol | | 20 | 22.5 | h | Wet + dry | |
| 38 | THF | | 10 | 18 | h | Wet + dry | |
| 39 | DMSO/MEK/Hexane | 1/40/25 | 66 | 11 | days | Wet + dry | |
| 40 | DMSO | | 2 | 6 | h | Wet + dry | |
| 41 | ACN | | 10 | 18 | | Wet + dry | |
| 42 | DMF/MEK | 5/15 | 20 | 72 | h | Wet + dry | MEK as anti solvent |
| 43 | DMA/MEK | 5/30 | 35 | 7 | days | Wet + dry | MEK as anti solvent |

### Examples 44 to 50

### Preparing ODV Succinate Form I Using a Slurry Method at Elevated Temperatures.

### General Process for Slurry at Different Temperature

To a 100 ml flask equipped with a magnetic stirrer and condenser were added ODV base and a solvent mixture. The mixture was stirred at ambient temperature for few minutes and then succinic acid (1.1 equivalent) was added. The mixture was stirred at ambient temperature and then cooled to room temperature until a solid precipitated. The solid was filtered under reduced pressure, washed and dried in a vacuum oven overnight at 50° C.

| | | | | Temp | | | | |
|---|---|---|---|---|---|---|---|---|
| | Solvent | Ratio | Vol | ° C. | Time | | Sample | Remarks |
| 44 | EtOAc/H₂O | 8/2 | 10 | 60 | 72 | h | Dry | |
| 45 | Hexane/H₂O | 8/2 | 10 | 60 | 72 | h | Dry | |
| 46 | CH₂Cl₂/H₂O | 8/2 | 10 | 60 | 72 | h | Dry | |
| 47 | Ethylene glycol/ | 5/25 | 30 | 65 | 6 | days | Wet | Hexane as anti solvent |
| | Hexane | | | | | | | |
| 48 | Di-ethylene glycol/ | 5/7 | 20 | 100 | 48 | h | Wet | Succinic acid diluted in |
| | H₂O | | | | | | | H₂O |
| 49 | H₂O | | 10 | 90 | 24 | h | wet | mixture was reheated and |
| | | | | | | | | cooled four times |
| 50 | THF | | 43 | 65 | 5.5 | h | wet | * Succinic acid diluted in |
| | | | | | | | | H₂O |
| | | | | | | | | * Hot filtration |

### Examples 51 and 52

### Preparing ODV Succinate Form I Using a Slurry in Melt Form.

### General Process for Slurry in Melt Form

To a 100 ml flask equipped with a magnetic stirrer were added ODV base and succinic acid (1.1 equivalent). The mixture was heated to melt material at the desired temperature and then a solvent was added. The mixture was stirred overnight at room temperature. The solid that precipitated was filtered under reduced pressure, washed and dried in a vacuum oven overnight at 50° C.

| | Solvent | Ratio | Vol | Temp | Time | | Sample |
|---|---|---|---|---|---|---|---|
| 51 | Iso amyl alcohol | | 10 | 130° C. | 16 | h | Wet |
| 52 | MIBK/H₂O | 10/0.5 | 10.5 | 110° C. | 4.5 | h | Wet |

### Examples 53 to 58

### Preparing ODV Succinate Form I from a Solution Using Sonication.

General Process for Sonication with 70% Amplitude

To a 100 ml beaker equipped with a magnetic stirrer were added at room temperature ODV base, a solvent mixture and succinic acid (1.1 equivalents). Sonication at 70% amplitude was started for few seconds or minutes. The temperature of the mixture rose to 50° C. and became a clear solution. Then the mixture was cooled and a solid precipitated. A sample was taken and in some experiments the suspension was stirred at room temperature. Then the solid was filtered under reduced pressure (only wet sample was analyzed).

| | | | | | | Time | | |
|---|---|---|---|---|---|---|---|---|
| | | | | Time | | stirring | | |
| | Solvent | Ratio | Vol | Sonication | | at RT | | Sample |
| 53 | H₂O | | 10 | 15 | min | | | Wet |
| 54 | IPA/H₂O | 10/2 | 12 | 15 | min | | | Wet |
| 55 | ACN/H₂O | 10/2 | 12 | 10 | min | | | Wet |
| 56 | Toluene/H₂O | 10/2 | 12 | 2.5 | min | 15 | min | Wet |
| 57 | EtOAc/H₂O | 10/2 | 12 | 10 | min | 16 | h | Wet |
| 58 | Di-iso propyl ether/ | 10/2 | 12 | 10 | min | 16 | h | Wet |
| | H₂O | | | | | | | |

### Examples 59 to 61

### Preparing ODV Succinate Form I Using Granulation.

### General Process for Granulation

To a 100 ml flask were added ODV succinate (200-300 mg) and MeOH (4-5 drops). The mixture was stirred at room temperature for 16 hours on evaporator, without vacuum and without heating. After 16 hours a sample was taken.

| | | | Starting | | |
|---|---|---|---|---|---|
| | solvent | vol | form | Time | sample |
| 59 | MeOH (200 mg) | 4 drops | form I | 16 h | Dry |
| 60 | MeOH (200 mg) | 4 drops | form II | 16 h | Dry |
| 61 | MeOH (300 mg) | 5 drops | form II | 16 h | Dry |

### Examples 62 to 71

### Preparing ODV Succinate Form I Using a Crystallization.

### General Process for Crystallization

To a 100 ml flask equipped with a magnetic stirrer were added ODV succinate salt and a solvent mixture. The mixture was heated to reflux. Whenever the solution was not clear, a hot filtration was performed and the filtrated mixture was transferred into a new clean flask equipped with a magnetic stirrer. Then the mixture was cooled in 30 minutes to room temperature while a heavy slurry precipitated. The mixture was further cooled to 5° C. in 30 minutes and for 30 minutes. Then the solid was filtered under reduced pressure and dried in a vacuum oven at 40° C. overnight.

| | Solvent | Ratio | Vol | Time | | Sample | Remarks |
|---|---|---|---|---|---|---|---|
| 62 | 1-PrOH/H₂O | 7/1 | 8 | 17 | h | Wet + dry | |
| 63 | t-BuOH | | 10 | 2 | h | Wet + dry | |
| 64 | IPA/H₂O | 4/2 | 6 | 1 | h | Wet + dry | |
| 65 | IPA/H₂O | 4/2 | 6 | 1 | h | wet | |
| 66 | 2-BuOH/H₂O | 4/2 | 6 | 1 | h | Dry | |
| 67 | 2-BuOH/H₂O | 4/2 | 6 | 4 | h | Wet | |
| 68 | ACN/H₂O | 4/2 | 6 | 1 | h | Wet | |
| 69 | MEK/H₂O | 4/2 | 6 | 4 | h | Wet | |
| 70 | IPA/H₂O | 4/2 | 6 | 7 | h | Wet | |
| 71 | EtOAc/MeOH | 4/2 | 6 | 3 | h | Wet | |

### Examples 72 to 77

### Preparing ODV Succinate Form I Using a Thermal Shock.

### General Process for Thermal Shocking

ODV succinate salt (5.7 g) was dissolved in MeOH (15 ml), 2.6 ml from this mixture was added into a pre-cooled (5° C.) flask containing a solvent (10 ml) and a magnetic stirrer. After stirring the mixture for 10-20 minutes a solid began to precipitate. The suspension was stirred at 5° C. for one hour. Then the solid was filtered under reduced pressure and dried in a vacuum oven at 40° C. overnight.

| | Solvent | Ratio | Vol | Sample | Remarks |
|---|---|---|---|---|---|
| 72 | Toluene | 10 | 12.6 | Wet + dry | ODVsucc in MeOH |
| 73 | Hexane | 10 | 12.6 | Wet + dry | ODVsucc in MeOH |
| 74 | EtOAc | 10 | 12.6 | Wet + dry | ODVsucc in MeOH |
| 75 | CH₂Cl₂ | 10 | 12.6 | Wet + dry | ODVsucc in MeOH |
| 76 | MTBE | 10 | 12.6 | Wet + dry | ODVsucc in MeOH |
| 77 | Acetone | 10 | 12.6 | Wet + dry | ODVsucc in MeOH |

### Example 78

A 100 ml three neck flask equipped with a mechanical stirrer, and a thermometer was charged with ODV base (2 g, 7.58 mmol) and water (20 ml), the suspension being stirred at room temperature. Succinic acid (1 g, 8.46 mmol) was added at room temperature and a clear solution was obtained. A heavy slurry precipitated after stirring for some time. Some of the material was filtered and the rest of it was stirred at room temperature overnight. Then the suspension was heated to 65° C. to obtain a clear solution and this solution was then cooled to room temperature, this heating-cooling sequence was repeated three times. The solid so obtained was then filtered under reduced pressure, washed and dried in a vacuum oven overnight at 50° C. to get ODV succinate form I.

### Preparation of ODV Succinate Salt Form II

### Examples 79 to 95

### Preparing ODV Succinate Form II Using a Slurry Method.

General process for slurry at room temperature

To a 100 ml flask equipped with a magnetic stirrer and condenser, were added ODV and a solvent mixture. The mixture was stirred at room temperature for few minutes and then succinic acid (1.1 equivalent) was added. The solution mixture was stirred at ambient temperature and a solid precipitated. Then the solid was filtered under reduced pressure, washed and dried in a vacuum oven overnight at 50° C.

| | Solvent | Ratio | Vol | Time | | Sample | Remark |
|---|---|---|---|---|---|---|---|
| 79 | H₂O | | 10 | 3 | days | Dry | 2 loops of heating at |
| | | | | | | | 65° C. and cooling at RT |
| 80 | H₂O +1% SLS (Sodium | | 12 | 3 | h | Wet | |
| | Loreal Sulfate) | | | | | | |
| 81 | Acetone/H₂O | 9/3 | | 12 | | Wet | |
| 82 | Acetone/H₂O | 1/1 | 4 | 2 | | Wet | |
| 83 | ACN/H₂O | 10/2 | 12 | 4 | h | Wet | |
| 84 | MeOH/H₂O/1% SLS | 10/2 | 10 | 1 | h | Wet | |
| 85 | Polyethylene glycol/H₂O | 10/2 | 12 | 16 | h | Wet | |
| 86 | EtOH abs | | 5 | 20 | | Wet + dry | |
| 87 | n-BuOH | | 5 | 20 | h | Dry | |
| 88 | 2-Ethoxyethanol | | 10 | 2 | h | Wet + dry | Cooled to 5° C. for 2 h |
| 89 | Dichloroethane | | 20 | 22.5 | h | Wet + dry | |
| 90 | BuOAc | | 20 | 22.5 | h | Wet + dry | |
| 91 | MeOAc | | 20 | 22.5 | h | Wet + dry | |
| 92 | EtOAc | | 7.5 | 20 | h | Wet | |
| 93 | DMC | | 10 | 20 | h | Wet + dry | |
| 94 | Ethyl lactate | | 5 | 20 | h | Wet + dry | |
| 95 | Saturated NaCl | | 10 | 16 | h | Wet + dry | Seeded with amorph form |

### Examples 96 to 98

### Preparing ODV Succinate Form II Using a Vapor Method.

General Process for Atmosphere of Form II with Heating

To a vial of 10 ml ODV succinate salt was added. This vial was put in a larger vial that contained a solvent and was closed with a septum. Then the vials were heated in a sand bath at 70° C. for 72 hours. Samples were dried in a vacuum oven at 50° C. overnight.

| | Solvent | Temp | Time | Remarks | Sample |
|---|---|---|---|---|---|
| 96 | | 70° C. | 72 h | Without solvent | Wet + dry |
| 97 | Acetone | 70° C. | 72 h | | Wet + dry |
| 98 | CH₂Cl₂ | 70° C. | 72 h | | Wet + dry |

### Examples 99 and 100

### Preparing ODV Succinate Form II Using Azaotropic Distillation.

### Process of Azeotropic Distillation

To a 100 ml flask equipped with a magnetic stirrer, a condenser and a dean stark were added at room temperature ODV base, succinic acid (1.1 equivalent) and a solvent mixture. The mixture was stirred and a solid precipitated at the ambient temperature. Then the suspension was heated to 80° C. and azeotropic distillation was performed. A solid precipitated after the mixture was stirred and cooled to room temperature for a certain time. The solid was filtered under reduced pressure and dried in a vacuum oven overnight at 50° C.

| | Solvent | Ratio | Vol | Temp | Time | | Sample |
|---|---|---|---|---|---|---|---|
| 99 | BuOH/H₂O | 8/2 | 10 | 80° C. | 2 | h | Dry |
| | | | | 5° C. | | | |
| | | | | RT | 16 | h | |
| 100 | Dioxane/H₂O | 10/2 | 12 | 80° C. | 2 | h | Dry |
| | | | | RT | 48 | h | |

### Examples 101 to 105

### Preparing ODV Succinate Form II Using Crystallization.

### General Process of Crystallization of ODV Succinate Salt

To a 100 ml flask equipped with a magnetic stirrer were added ODV succinate salt and a solvent mixture. The mixture was heated to reflux. Whenever the solution was not clear, a hot filtration was performed and the filtrated mixture was transferred into a new clean flask equipped with a magnetic stirrer. Then the mixture was cooled in 30 minutes to room temperature while a heavy slurry precipitated. The mixture was further cooled to 5° C. in 30 minutes and for 30 minutes. Then the solid was filtered under reduced pressure and dried in a vacuum oven at 40° C. overnight.

| | Solvent | Vol | Time | Sample | Remarks |
|---|---|---|---|---|---|
| 101 | n-BuOH | 10 | 2 h | Dry | Starting material form II |
| 102 | 2-BuOH | 10 | 2 h | Wet + dry | Starting material form II |
| 103 | 1-PrOH | 7 | 2 h | Wet + dry | Starting material form II |
| 104 | MIBK | 10 | 2 h | Wet + dry | Starting material form II |
| 105 | dichloro- | 15 | 2 h | Wet + dry | Starting material form II |
| | benzene | | | | |

### Example 106

### Preparing ODV Succinate Form II Using a Sonication.

General Process for Sonication with 40% Amplitude

To a beaker at room temperature were added ODV base, water and methylethylketone and the mixture stirred for 9.3 minutes. Succinic acid (1.1 equivalent) was then added and a clear solution was obtained. Sonication was started for 18 minutes with 40% amplitude (5 seconds on and 15 seconds off) and a solid precipitated. A sample was taken and the rest of the mixture was kept at the same conditions for 40 minutes. Then the solid was filtered under reduced pressure.

| | Solvent | Ratio | Vol | Time | Sample |
|---|---|---|---|---|---|
| 106 | MEK/H₂O | 10/2 | 12 | 18 min | Wet |

### Example 107

### Preparing ODV Succinate Form II Using Crystallization.

### Process for Crystallization

To a 25 ml flask equipped with a magnetic stirrer and a condenser were added succinic acid (1.1 equivalents) and water. Then the mixture was heated to 55° C. in an oil bath and the mixture became clear. At 55° C., ODV base was added slowly in portions and a solid precipitated slowly. The suspension was cooled to 30° C. in 15 minutes in the oil bath, kept at 30° C. for 15 minutes and then cooled to 50° C. for 30 minutes. Then the solid was filtered, washed with water and dried in a vacuum oven at 40° C. overnight.

| | solvent | Vol | Temp (° C.) | Time | Sample |
|---|---|---|---|---|---|
| 107 | H₂O | 1.5 | 55 | 1 h | Wet + dry |

### Examples 108 to 109

### Preparing ODV Succinate Form II Using Crystallization.

### General Process of Crystallization of ODV Succinate Salt

To a 100 ml flask equipped with a magnetic stirrer were added ODV succinate salt and a solvent mixture. The mixture was heated to reflux. Whenever the solution was not clear, a hot filtration was performed and the filtrated mixture was transferred into a new clean flask equipped with a magnetic stirrer. Then the mixture was cooled in 30 minutes to room temperature while a heavy slurry precipitated. The mixture was further cooled to 5° C. in 30 minutes and for 30 minutes. Then the solid was filtered under reduced pressure and dried in a vacuum oven at 40° C. overnight.

| | Solvent | Vol | Time | Sample | Remarks |
|---|---|---|---|---|---|
| 108 | EtOH | 25 | 2 h | Wet | Start material form II |
| 109 | ACN | 15 | 2 h | Wet + dry | Start material form II |
| 110 | Dioxane | 15 | 2 h | Wet + dry | Start material form II |

### Preparation of ODV Succinate Salt Form III

### Examples 111 to 112

### Preparing ODV Succinate Form III Using a Slurry Method.

### General Process for Slurry at Different Temperatures

To a 100 ml flask equipped with a magnetic stirrer and a condenser, were added ODV base and a solvent. The mixture was stirred at room temperature for few minutes and then succinic acid (1.1 equivalent) was added. The mixture was stirred at room temperature and a solid precipitated. Then the solid was filtered under reduced pressure, washed and dried in a vacuum oven overnight at 50° C.

| | Solvent | Ratio | Vol | Time | | Temp | Sample |
|---|---|---|---|---|---|---|---|
| 111 | EtOH/Ether | 28/8 | 36 | 24 | h | | Wet |
| 112 | Di-iso propyl | | 7.5 | 18 | h | | wet |
| | ether | | | | | | |
| 113 | Butyl lactate | | 6 | 16 | h | | Dry |
| 114 | CH₂Cl₂ | | 15 | 18 | h | | Wet + |
| | | | | | | | dry |
| 115 | Toluene/H₂O/0.1 | 8/2 | 10 | 6 | days | | Wet |
| | eq aliquat 366 | | | | | | |
| 116 | No solvent | | | 54.5 | h | 100° C. | dry |
| | | | | | | to RT | |

*Example 116: ODV succinate salt was heated to melt form at 80-100° C., then to room temperature

### Examples 117 to 120

### Preparing ODV Succinate Form III Using Pressure.

Process of Press, Form I+II

A disk plate was prepared from ODV succinate salt mixture of forms I+II and solvent (3-4 drops), by pressing under 10 tons the solid material in the CARVER laboratory press for 30 minutes to 2 hours.

| | Solvent | Time | | sample | Remarks |
|---|---|---|---|---|---|
| 117 | | 2 | h | Dry | Without solvent |
| 118 | H₂O | 30 | min | Dry | |
| 119 | MeOH | 30 | min | Dry | |
| 120 | Acetone | 30 | min | Dry | |

### Example 121

### Preparing ODV Succinate Form III Using a Azeotropic Distillation.

### Process of Azeotropic Distillation

To a 100 ml flask equipped with a magnetic stirrer, condenser and dean stark were added at room temperature ODV base, succinic acid (1.1 equivalent) and a solvent mixture. The mixture was stirred and a solid precipitated at ambient temperature. Then the suspension was heated to 80° C. and azeotropic distillation was performed. An oily material was obtained in the flask. After stirring at room temperature for 24 h, MEK was added. A solid precipitated after 9 days stirring at room temperature. Then the solid was filtered under reduced pressure and dried in a vacuum oven overnight at 500° C.

| | Solvent | Ratio | Vol | Time | Sample |
|---|---|---|---|---|---|
| 121 | MEK/H₂O | 10/2 | 12 | 9 days | Dry |

### Examples 122 to 123

### Preparing ODV Succinate Form III Using a Milling Method.

### General Process for Milling of Form I+II

In a mortar were added ODV succinate salt (300 mg) and 3 drops of a solvent, the mixture was milled in a mortar for 1 hour at room temperature.

| | Solvent | Vol | Time | Sample |
|---|---|---|---|---|
| 122 | H₂O | 3 drops | 1 h | Dry |
| 123 | MeOH | 3 drops | 1 h | Dry |

### Preparation of ODV Succinate Salt Form IV

### Example 124

### Preparing ODV Succinate Form IV Using a Vapor Method.

General Process for Atmosphere of Form II with Heating

To a vial of 10 ml ODV succinate salt was added. This vial was put in a larger vial that contained a solvent and was closed with a septum. Then the vials were heated in a sand bath at 70° C. for 72 hours. Samples were dried in a vacuum oven at 50° C. overnight.

| | Solvent | Time | Remarks |
|---|---|---|---|
| 124 | MeOH | 72 h | |

### Examples 125 to 128

### Preparing ODV Succinate Form IV Using a Drying Method.

### Hygroscopicity

To an oven preheated to 80° C. was inserted a dessicator containing ODV succinate salt in a crystallizator dish and solid P₂O₅.

| | | |
|---|---|---|
| Start form | Time | Humidity |

| | | | | |
|---|---|---|---|---|
| 125 | II | 1 | week | 0% RH |
| 126 | II | 17 | days | 0% RH |
| 127 | I | 1 | week | 0% RH |
| 128 | I | 17 | days | 0% RH |

### Preparation of ODV Succinate Salt Form I+II

### Examples 129 to 135

### Preparing ODV Succinate Forms I and II Using a Slurry Method.

### General Process for Slurry at Room Temperature

To a 100 ml flask equipped with a magnetic stirrer and a condenser, were added ODV base and a solvent mixture. The mixture was stirred at room temperature for few minutes and then succinic acid (1.1 equivalent) was added. The mixture was stirred at room temperature and a solid precipitated. Then the solid was filtered under reduced pressure, washed and dried in a vacuum oven overnight at 50° C.

| | Solvent | Ratio | Vol | Time | | Sample |
|---|---|---|---|---|---|---|
| 129 | H₂O/SLS 1% | 12 | 12 | 16 | h | wet |
| 130 | MTBE | | 10 | 22.5 | | |
| 131 | n-Amyl alcohol | | 5 | 22.5 | h | |
| 132 | Cellosolve | | 5 | 22.5 | h | |
| | (2-ethoxyethanol) | | | | | |
| 133 | Iso BuOAc | | 7.5 | 22.5 | h | |
| 134 | Diethylenglycol/ | 5/15 | 20 | 15 | h | Dry |
| | MEK | | | | | |
| 135 | Di-ethyl Ether | | 20 | 16 | h | Dry |

### Examples 136 and 137

### Preparing ODV Succinate Forms I and II Using a Azeotropic Distillation.

### General Process of Azeotropic Distillation

To a 100 ml flask equipped with a magnetic stirrer and dean stark were added at room temperature ODV base, succinic acid (1.1 equivalents) and a solvent mixture. The mixture was stirred and a solid precipitated at ambient temperature. Then the suspension was heated to 80-105° C. and azeotropic distillation was performed. A solid or an oily material was obtained in the flask. The mixture was stirred and cooled to room temperature. Then the solid was filtered under reduced pressure and dried in a vacuum oven overnight at 50° C.

| | | | | Temp | | | |
|---|---|---|---|---|---|---|---|
| | Solvent | Ratio | Vol | (° C.) | Time | | Remarks |
| 136 | IPA/H₂O | 7/3 | 10 | 85-105 | 5 | h | Wet |
| | | | | RT | 16 | h | |
| 137 | CHCl₃/H₂O | 10/2 | 12 | 80 | 1.5 | h | Dry |

| | |
|---|---|
| RT | 2.5 |

### Example 138

### Preparing ODV Succinate Form I and II Using Crystallization.

### Process of Crystallization

To a flask equipped with a magnetic stirrer and a condenser were added ODV base (1 g, 3.79 mmol) and acetone (38 ml), then the mixture was heated to 55° C. At this temperature a solution mixture of succinic acid (1 g, 8.47mmol) in acetone (30 vol) was added dropwise. The mixture was stirred for 30 minutes and a hot filtration was performed. The heating was preformed for another 2 hours at 55° C., then the mixture was cooled to 0-5° C. for 2 hours. Then the solid was filtered, washed with cold acetone.

| | Solvent | Vol | Time | Remarks |
|---|---|---|---|---|
| 138 | Acetone | 38 | 2 h | Wet |

### Preparation of ODV Succinate Salt Form I>II

### Examples 139 to 142

### Preparing ODV Succinate Form I>II Using a Slurry Method.

General process for slurry

To a 100 ml flask equipped with a magnetic stirrer and a condenser, were added ODV and solvent mixture. The mixture was stirred at room temperature for few minutes and then succinic acid (1.1 equivalent) was added. The mixture was stirred at ambient temperature and a solid precipitated. Then the solid was filtered under reduced pressure, washed and dried in a vacuum oven overnight at 50° C.

| | Solvent | Ratio | Vol | Time | | Temp | Sample |
|---|---|---|---|---|---|---|---|
| 139 | Ethylene | 5/25 | 30 | 16 | h | 65° C. | Dry |
| | glycol/ | | | 5 | days | RT | |
| | hexane | | | | | | |
| 140 | MEK | | 5 | 20 | h | RT | Wet |
| 141 | 2-BuOH | | 5 | 20 | h | RT | Wet |
| 142 | n-BuOH/ | 8/2 | 10 | 16 | h | RT | Wet |
| | MeOH | | | | | | |

### Example 143

### Preparing ODV Succinate Forms I>II Using Sonication.

Process of Sonication with 70% Amplitude )

To a 100 ml beaker equipped with a magnetic stirrer, were added heptane, water, succinic acid (1.1 equivalent) and ODV base, the suspension was put under sonication for 10 minutes with 70% amplitude. The suspension was stirred for 16 hours at room temperature. A sample was analyzed after filtration.

| | Solvent | Ratio | Vol | Time | Temp | Sample |
|---|---|---|---|---|---|---|
| 143 | n-Heptane/H₂O | 10/2 | 12 | 10 min | RT | wet |

### Example 144

### Preparing ODV Succinate Forms I>II Using Crystallization.

### Process of Crystallization

To a flask equipped with a magnetic stirrer and a condenser were added ODV base, acetonitrile and water (9:1) stirring at room temperature. To the suspension succinic acid (1.1 equivalent) was added and the mixture was heated to reflux. The mixture became clear and more water (10 ml) was added. The mixture was cooled slowly to 30° C. in the oil bath; at 50° C. the mixture was seeded with form II, and then continued cooling to 5-0° C. for 3 hours. Then the solid was filtered, washed with water and dried in vacuum oven at 40° C. overnight.

| | Solvent | Ratio | Vol | Time | Sample |
|---|---|---|---|---|---|
| 144 | ACN/H₂O | 9/2 | 11 | 4 h | Wet + dry |

### Preparation of ODV Succinate Salt Form II>I

### Examples 145 to 149

### Preparing ODV Succinate Forms II>I Using a Slurry Method.

### Process of Slurry at Room Temperature

To a 100 ml flask equipped with a magnetic stirrer and a condenser, were added ODV base and a solvent mixture. The mixture was stirred at room temperature for few minutes and then succinic acid (1.1 equivalent) was added. The suspension was stirred at ambient temperature. Then the solid was filtered under reduced pressure, washed and dried in a vacuum oven at 50° C. overnight.

| | Solvent | Ratio | Vol | Temp | Time | | Sample |
|---|---|---|---|---|---|---|---|
| 145 | Acetone/H₂O | 2/2 | 4 | RT | 1 | h | wet |
| | | | | 0-5° C. | 1 | h | |
| 146 | Dioxane/H₂O | 10/2 | 12 | | 24 | h | wet |
| 147 | EtOAc/MeOH | 10/2 | 12 | | 16 | h | Wet |
| 148 | Poly Ethylene | 10/2 | 12 | | 16 | h | Dry |
| | glycol/MeOH | | | | | | |
| 149 | Dioxane/MeOH/ | 6/2/2 | 10 | | 48 | h | Wet + dry |
| | H₂O | | | | | | |

### Example 150

### Preparing ODV Succinate Forms II>I Using a Vapor Method.

General Process for Atmosphere of Form II with Heating

To a vial of 10 ml ODV succinate (form II) salt was added. The vial was placed in a larger vial that contained a solvent and was closed with septum. The vials were heated in a sand bath at 70° C. for 72 hours. The solid was dried in vacuum oven at 50° C. overnight.

| | Solvent | Temp | Time | Sample |
|---|---|---|---|---|
| 150 | H₂O | 70° C. | 72 h | Wet + dry |

### Preparation of ODV Succinate Salt Form II+III

### Example 151

A 100 ml three necks flask equipped with a mechanical stirrer, condenser and a thermometer, was charged with ODV base (1 g, 3.79 mmol) and methyisobutyl ketone (10 ml), this suspension being stirred and heated to 65° C. Succinic acid (0.5 g, 4.23 mmol) was added and the mixture was stirred overnight at 65° C., then cooled to room temperature and stirred overnight at room temperature. The solid was filtered under reduced pressure, washed and dried at 50° C. under vacuum overnight to get ODV succinate form II+III.

### Example 152

A 100 ml three necks flask equipped with a mechanical stirrer, condenser and a thermometer, was charged with ODV base (1 g, 3.79 mmol) and n-Heptane (10 ml), this suspension being stirred and heated to 65° C. Succinic acid (0.5 g 4.23 mmol) was added and the mixture was stirred at 65° C. overnight, then the mixture was cooled to room temperature and stirred overnight at room temperature. The solid was filtered under reduced pressure, washed and dried at 50° C. under vacuum overnight to get ODV succinate form II+III.

### Preparation of ODV Succinate Salt Form IV+II

### Examples 153 to 155

### Preparing ODV Succinate Form IV and II Using a Slurry Method.

### General Process for Slurry

To a 100 ml flask equipped with a magnetic stirrer and condenser, were added ODV base and the solvent mixture. The mixture was stirred at room temperature for few minutes and succinic acid (1.1 equivalent) was added. The mixture was stirred at room temperature. Then the solid was filtered under reduced pressure, washed and dried in a vacuum oven overnight at 50° C.

| | Solvent | Ratio | Vol | Time | Temp | Sample |
|---|---|---|---|---|---|---|
| 153 | EtOAc/MeHO | 8/2 | 10 | 72 h | RT | Wet |
| 154 | CH₂Cl₂ | | 10 | 72 h | 65° C. | Wet |
| 155 | IPA/MeOH | 8/2 | 10 | 72 h | 65° C. | Wet |

Preparation of ODV succinate salt form IV>II

### Example 156

### Preparing ODV Succinate Form IV>II Using a Slurry Method.

### General Process for Slurry

To a 100 ml flask equipped with a magnetic stirrer and condenser, were added ODV base and the solvent mixture. The mixture was stirred at room temperature for few minutes and succinic acid (1.1 equivalent) was added. The mixture was stirred at room temperature. Then the solid was filtered under reduced pressure, washed and dried in a vacuum oven overnight at 50° C.

| | Solvent | Ratio | Vol | Time | Temp | Sample |
|---|---|---|---|---|---|---|
| 156 | EtOAc/MeOH | 8/2 | 10 | 72 h | RT | Dry |

### Preparation of ODV Succinate Salt Amorphous Form

### Examples 157 to 159

### Preparing Amorphous ODV Succinate Using Drying Method.

### General Process of Drying in Vacuum Oven at 150° C.

To a crystallizator dish ODV succinate salt was weighted and 3 drops of water were added. The solid was dried in a vacuum oven at 150° C. overnight.

| | Form | Solvent | Time | Temp (° C.) | Sample |
|---|---|---|---|---|---|
| 157 | I + II | H₂O | 16 h | 150 | Dry |
| 158 | I | H₂O | 16 h | 150 | Dry |
| 159 | II | H₂O | 16 h | 150 | Dry |

### Example 160

### Preparing Amorphous ODV Succinate.

Process for Preparation Amorphous Form without Solvent

To a flask equipped with a magnetic stirrer were weighted ODV base and succinic acid (1.1 equivalents). The mixture was heated to 80° C. and then to 100° C. overnight, the solid material turned into melt. Then the melt material was cooled to room temperature and a sample was analyzed.

| | Solvent | Temp (° C.) | Time | Sample |
|---|---|---|---|---|
| 160 | Solid/solid | 100 | 16 h | Dry |

### Example 161

### Preparing Amorphous ODV Succinate.

### Lyophilization

ODV succinate (760 mg) was dissolved in t-butanol (20 ml) by heating. The solution was allowed to cool to ambient temperature (15 min) and that was frozen by liquid nitrogen. Lyophilisation was carried out at Lyovac GT-2 (Leybold-Heareus) instrument at 1 mBar. Product was light white powder.

### Examples 162 and 163

### Preparing Amorphous ODV Succinate by Precipitation.

### Process of Precipitation

To a flask equipped with a magnetic stirrer and a condenser were added ODV base (1 g, 3.79 mmol) and alcohol (38 ml), then the mixture was heated to 55° C. At this temperature a solution mixture of succinic acid (1 g, 8.47 mmol) in alcohol (30 vol) was added dropwise. The mixture was stirred for 30 minutes and a hot filtration was performed. The heating was preformed for another 2 hours at 55° C., then the mixture was cooled to 0-5° C. for 2 hours. Then the solid was filtered, washed with cold alcohol.

| | Solvent | Vol | Time | Sample |
|---|---|---|---|---|
| 162 | EtOH | 25 | 2 h | Dry |
| 163 | MeOH | 4 | 2 h | Wet + dry |

### Examples 164 to 167

### Preparing Amorphous ODV Succinate.

### General Process of Spray Dryer

To a 250 ml reactor were added at room temperature ODV succinate salt and a solvent. The mixture was heated to reflux to obtain almost clear solution. A hot filtration was performed to remove insoluble particles. The clear solution was transferred back into the reactor and heated again to reflux. Then the solution was sprayed into the chamber of a spray dryer apparatus with ambient nitrogen at co-current flow. The atomizing flow (660 [1/h]) of nitrogen produced droplets which led to high evaporation rate.

The obtained sample was analyzed.

| | | | | | | | Spray | Feed | | |
|---|---|---|---|---|---|---|---|---|---|---|
| | | | | Temp | Temp | | N₂ | rate | Nozzle | |
| | Solvent | Vol | Time | ° C. (in) | ° C. (out) | Aspirator | [m³/h] | [ml/h] | clear | sample |
| 164 | EtOH | 10 | 10 min | 80 | 40 | 100 | 40 | 20 | 2 | Dry |
| 165 | MeOH | 10 | 10 min | 80 | 40 | 100 | 40 | 20 | 2 | Dry |
| 166 | MeOH | 10 | 40 min | 78 | 47 | 90 | 30 | 20 | 2 | Dry |
| 167 | EtOH | 10 | 40 min | 90 | 40 | 95 | 30 | 20 | 2 | Dry |

### Preparation of ODV Succinate Salt Form A (ODV Base)>II

### Examples 168 and 169

### Preparing ODV Succinate Form A>II Using a Slurry Method.

### Process of Slurry at Room Temperature

To a 100 ml flask equipped with a magnetic stirrer and a condenser, were added ODV base and a solvent mixture. The mixture was stirred at room temperature for few minutes and then succinic acid (1.1 equivalent) was added. The mixture was stirred at this temperature for a certain time. Then the solid was filtered under reduced pressure, washed and dried in a vacuum oven overnight at 50° C.

| | Solvent | Ratio | Vol | Time | | Sample |
|---|---|---|---|---|---|---|
| 168 | DCB | | 5 | 22.5 | h | Dry |
| 169 | DMSO/H₂O | 8/2 | 10 | 4 | days | wet |

### Examples 170 to 174

### Preparing ODV Succinate Form A>II.

Atmosphere with Heating Base+Succinic Acid

To a vial of 10 ml were added ODV base and succinic acid. The vial was placed in a larger vial that contained a solvent and closed with a septum. Then these vials were heated in a sand bath at 70° C. for 72 hours. The samples were dried in a vacuum oven at 50° C. overnight.

| | Solvent | Time | Remarks |
|---|---|---|---|
| 170 | | 72 h | Without solvent |
| 171 | MeOH | 72 h | |
| 172 | H₂O | 72 h | |
| 173 | Acetone | 72 h | |
| 174 | CH₂Cl₂ | 72 h | |

What is claimed is:

1. A process of preparing crystalline O-desmethylvenlafaxine succinate form I comprising: maintaining a slurry or suspension comprising O-desmethylvenlafaxine, succinic acid and a solvent for a sufficient time to form crystalline O-desmethylvenlafaxine succinate form I, wherein the solvent is selected from the group consisting of: water; a mixture of water and tween, dimethylsulfoxide (DMSO), dimethyacetamide (DMA), toluene, dioxane, butanol, dichloromethane (DCM), hexane, ethylacetate, cyclohexanone and/or methanol; methanol; a mixture of methanol and hexane, cyclohexanone, acetonitrile, methylethyl ketone (MEK), toluene, acetone, dioxane and/or xylene; a mixture of water, methanol and isopropylalcohol, acetonitrile, MEK, toluene, 2-butanol and/or acetone; 1-propanol; t-butanol; 1-octanol; a mixture of isopropyl alcohol, acetic acid and heptane; iso amyl alcohol; tetrahydrofuran (THF); DMSO; a mixture of DMSO, MEK, and hexane; acetonitrile; a mixture of dimethylformamide (DMF) and MEK; and a mixture of DMA and MEK.

2. The process of claim 1, further comprising a phase transfer catalyst.

3. The process of claim 2, wherein the catalyst is aliquat 366 or an equivalent thereof.

4. A process of preparing crystalline O-desmethylvenlafaxine succinate form I comprising: maintaining a slurry or suspension comprising O-desmethylvenlafaxine, succinic acid and a solvent for a sufficient time at a temperature of about 60° C. to about 100° C. to form crystalline O-desmethylvenlafaxine succinate form I, wherein the solvent is selected from the group consisting of: water; a mixture of water and ethylacetate, hexane, DCM and/or diethylene glycol; THF; and a mixture of ethylene glycol and hexane, wherein when the solvent is water, the temperature is about 90° C.

5. A process for preparing crystalline O-desmethylvenlafaxine succinate form I comprising: combining O-desmethylvenlafaxine, succinic acid and a solvent to form a mixture; and precipitating O-desmethylvenlafaxine form I from the mixture.

6. The process of claim 5, wherein the O-desmethylvenlafaxine and succinic acid are combined and melted together to form a melt, followed by combining the melt with the solvent.

7. The process of claim 5, wherein the solvent is a C₅₋₈ alcohol or a mixture of a C₄₋₇ ketone and water.

8. A process for preparing crystalline O-desmethylvenlafaxine succinate form I comprising: providing a solution of O-desmethylvenlafaxine succinate in a solvent selected from the group consisting of water; a mixture of water and isopropyl alcohol, acetonitrile, toluene, ethylacetate, di-isopropylether, 1-propanol, t-butanol, 2-butanol, and/or MEK; and a mixture of methanol and ethylacetate; and precipitating crystalline O-desmethylvenlafaxine succinate form I from the solution, wherein when the solvent is a mixture of water and MEK, the mixture is in a 2:4 ratio.

9. The process of claim 8, wherein prior to the precipitating step, the solution is heated to a temperature above room temperature; and wherein the precipitating step comprises a first step of cooling the heated solution to about room temperature and a second step of cooling the solution to about −5° C. to about 15° C.

10. A process preparing crystalline O-desmethylvenlafaxine succinate comprising:
providing a solution of O-desmethylvenlafaxine succinate in a C₁₋₄ alcohol; and
precipitating crystalline O-desmethylvenlafaxine succinate form I by cooling the solution to a temperature of about −5° C. to about 15° C.

11. The process of claim 10, wherein the cooling step comprises adding the solution to a pre-cooled solvent selected from the group consisting of a C₆₋₈ aromatic hydrocabon, a C₆₋₈ hydrocarbon, a C₄₋₇ ester, a halogenated C₁₋₄ hydrocarbon, a C₃₋₈ ether, and acetone.

12. The process of claim 11, wherein the solvent is pre-cooled to about −5° C. to about 15° C.

13. A process of preparing crystalline O-desmethylvenlafaxine succinate form II comprising: combining O-desmethylvenlafaxine, succinic acid and a solvent selected from the group consisting of: a mixture of water and a phase transfer catalyst, acetone, acetonitrile, polyethylene glycol and/or methanol and a phase transfer catalyst; ethanol; n-butanol; 2-ethoxyethanol; dichloroethane; buthylacetate; methylacetate; ethylacetate; dimethylcarbonate; ethyl lactate and saturated sodium chloride in water; to form a reaction mixture; and crystallizing O-desmethylvenlafaxine succinate form II from the reaction mixture.

14. The process of claim 13, wherein the reaction mixture includes sodium laurel sulfate (SLS).

15. A process of preparing crystalline O-desmethylvenlafaxine succinate form II comprising: heating a mixture of O-desmethylvenlafaxine, succinic acid and water to a temperature of about 60° C. to about 70° C.; and crystallizing O-desmethylvenlafaxine form II from the heated mixture.

16. A process for preparing crystalline O-desmethylvenlafaxine succinate form II comprising exposing O-desmethylvenlafaxine succinate to solvent vapors for a period of time sufficient to obtain crystalline O-desmethylvenlafaxine succinate form II.

17. The process of claim 16, wherein the solvent is acetone or a halogenated C₁₋₄ hydrocarbon.

18. A process for preparing crystalline O-desmethylvenlafaxine succinate form II comprising heating O-desmethylvenlafaxine succinate in a closed environment in the absence of a solvent.

19. A process for preparing crystalline O-desmethylvenlafaxine succinate form II comprising: providing a mixture of O-desmethylvenlafaxine base, succinic acid, and a solvent; and removing the solvent to obtain crystalline O-desmethylvenlafaxine succinate form II,
wherein the solvent is selected from a mixture of water with a C₁₋₄ alcohol; or a C₄₋₈ cyclic ether.

20. The process of claim 19, wherein the removing step comprises azeotropic distillation.

21. A process for preparing crystalline O-desmethylvenlafaxine form II comprising:
providing a solution of O-desmethylvenlafaxine succinate in a solvent selected from the group consisting of n-butanol; 2-butanol; 1-propanol; ethanol; methyl isobutyl ketone (MIBK); dichlorobenzene; a mixture of water and methylethyl ketone (MEK); acetonitrile; and dioxane; and precipitating crystalline O-desmethylvenlaxine succinate form II from the solution,
wherein when the solvent is acetonitrile, the precipitating step is carried out for about 1 hour to about 4 hours.

22. The process of claim 21, wherein the providing step comprises (a) heating a mixture of O-desmethylvenlafaxine succinate and the solvent or (b) combining succinic acid with a mixture of O-desmethylvenlafaxine base, water and a C₄₋₇ ketone.

23. A process preparing O-desmethylvenlafaxine succinate form II comprising:
providing a mixture of succinic acid and water; heating the mixture; and
adding O-desmethylvenlafaxine base to the heated mixture; and cooling the heated mixture to obtain crystalline O-desmethylvenlafaxine succinate form II.

24. A process preparing O-desmethylvenlafaxine succinate form III comprising:
combining O-desmethylvenlafaxine, succinic acid and a solvent selected from the group consisting of a mixture of ethanol and ether; di-isopropyl ether; butyl lactate; DCM; and a mixture of water, toluene and a phase transfer catalyst; to form a reaction mixture; and crystallizing O-desmethylvenlafaxine succinate form III from the reaction mixture.

25. A process preparing O-desmethylvenlafaxine succinate form III comprising:
reacting O-desmethylvenlafaxine and succinic acid at a temperature of about 80 to about 100° C. for more than 50 hours; and cooling to room temperature to obtain crystalline O-desmethylvenlafaxine Form III.

26. A process preparing crystalline O-desmethylvenlafaxine succinate form III comprising:
exposing crystalline forms I and II of O-desmethylvenlafaxine succinate under sufficient pressure to obtain crystalline O-desmethylvenlafaxine form III.

27. The process of claim 26, further comprising adding about 3-5 drops of solvent per about 300 grams of crystalline forms I and II of O-desmethylvenlafaxine prior to subjecting it to pressure.

28. The process of claim 27, wherein the solvent is selected from the group consisting of water, a C₁₋₄ alcohol, and a C₃₋₇ ketone.

29. A process for preparing crystalline O-desmethylvenlafaxine succinate form III comprising: providing a mixture of O-desmethylvenlafaxine base, succinic acid and a solvent; and removing the solvent to obtain an oily substance; adding Methyl Ethyl Ketone (MEK) to the oily substance to form a mixture; and maintaining the mixture for a period of time sufficient to obtain crystalline O-desmethylvenlafaxine form III.

30. The process of claim 29, wherein the solvent is a mixture of a C₄₋₇ ketone and in water.

31. The process of claim 29, wherein the removing step comprises azeotropic destillation.

32. A process for preparing crystalline O-desmethylvenlafaxine succinate form III comprising: milling O-desmethylvenlafaxine with about 1 drop of water or methanol per 100 mg O-desmethylvenlafaxine.

33. A process preparing crystalline O-desmethylvenlafaxine succinate form IV comprising: exposing O-desmethylvenlafaxine succinate to vapors of a C₁₋₄ alcohol to obtain crystalline O-desmethylvenlafaxine form IV.

34. A process preparing crystalline O-desmethylvenlafaxine succinate forms IV comprising: drying crystalline O-desmethylvenlafaxine succinate form I or II for a period of time sufficient to obtain crystalline O-desmethylvenlafaxine form IV.

35. A process preparing a mixture of crystalline O-desmethylvenlafaxine succinate forms I and II comprising: combining O-desmethylvenlafaxine, succinic acid and a solvent selected from the group consisting of: a mixture of water and 1% sodium laurel sulfate (SLS); methyl tertbutyl ether (MTBE); n-amyl alcohol; 2-ethoxyethanol (cellosolve); isobutylacetate; a mixture of diethyleneglycol and methyl ethyl ketone (MEK); di-ethyl ether; a mixture of ethylene glycol and hexane; methyl ethyl ketone (MEK); 2-butanol; a mixture of n-butanol and methanol; a mixture of water and either acetone, dioxane or dioxane and methanol; and a mixture of methanol and either ethylacetate or polyethyleneglycol; to form a reaction mixture; and
crystallizing a mixture of O-desmethylvenlafaxine succinate forms I and II from the reaction mixture, wherein when the solvent is 2-ethoxyethanol, the crystallizing step comprises maintaining the reaction mixture for a period of about 22 hours, and when the solvent is a mixture of water and 1% SLS, the crystallizing step comprises maintaining the reaction mixture for a period of about 16 hours.

36. A process preparing a mixture of crystalline O-desmethylvenlafaxine succinate forms I and II comprising: providing a mixture of O-desmethylvenlafaxine base, succinic acid, and a solvent; and removing the solvent to obtain crystalline O-desmethylvenlafaxine succinate forms I and II, wherein the solvent is a mixture of water with isopropyl alcohol or chloroform.

37. A process preparing a mixture of crystalline O-desmethylvenlafaxine succinate forms I and II comprising: providing a mixture of O-desmethylvenlafaxine base and acetone; heating the mixture; adding succinic acid and acetone to the heated mixture; maintaining the heated mixture for about 1 hour to about 4 hours; and cooling the heated mixture to obtain a mixture of crystalline O-desmethylvenlafaxine forms I and II.

38. A process of preparing a mixture of crystalline O-desmethylvenlafaxine succinate forms I and II comprising: providing a suspension of O-desmethylvenlafaxine base, acetonitrile and water; adding succinic acid to the suspension to obtain a mixture; heating the mixture to obtain a solution; adding water to the solution; cooling slowly to about 30° C.; and cooling further to a temperature of about 0-5° C. to obtain a mixture of crystalline O-desmethylvenlafaxine forms I and II.

39. A process preparing a mixture of crystalline O-desmethylvenlafaxine succinate forms I and II comprising: providing O-desmethylvenlafaxine base and succinic acid in a solvent to obtain a suspension; sonicating the suspension; and maintaining the suspension for a period of time sufficient to obtain a mixture of crystalline O-desmethylvenlafaxine forms I and II, wherein the solvent mixture is a mixture of water with a C₆₋₈ hydrocarbon.

40. A process preparing a mixture of crystalline O-desmethylvenlafaxine succinate forms I and II comprising: exposing Form II to water vapors.

41. A process preparing amorphous O-desmethylvenlafaxine succinate comprising:
reacting O-desmethylvenlafaxine and succinic acid at a temperature of about 80-100° C. for about 16 hours; and cooling to room temperature to obtain amorphous O-desmethylvenlafaxine.

42. A process preparing amorphous O-desmethylvenlafaxine succinate comprising:
dissolving O-desmethylvenlafaxine in a C₁₋₄ alcohol; and removing the solvent by lyophilizing or spray drying the solution to obtain amorphous O-desmethylvenlafaxine.

43. A process preparing amorphous O-desmethylvenlafaxine succinate comprising:
providing a mixture of O-desmethylvenlafaxine base and a C₁₋₄ alcohol; heating the mixture; adding a solution of succinic acid in a C₁₋₄ alcohol to the heated mixture; maintaining the heated mixture for about 1 hour to about 4 hours; and cooling the heated mixture to obtain amorphous O-desmethylvenlafaxine.

44. A process preparing amorphous O-desmethylvenlafaxine succinate comprising:
providing solid O-desmethylvenlafaxine succinate and about 2 to 6 drops of water per 300 gram of O-desmethylvenlafaxine succinate; and drying the solid at a temperature of about 120° C. to about 175° C. for a period of time sufficient to obtain amorphous O-desmethylvenlafaxine.
